(19) United States
(12) Patent Application Publication
(10) Pub. No.: US 2004/0216177 A1
(43) Pub. Date: Oct. 28, 2004

(54) Congenic rats containing a mutant GPR10 gene

(75) Inventors: Shaun Jordan, Frederick, MD (US); Akihito Watanabe, Tokushima-city (JP); Shiro Okuno, Tokushima-city (JP); Takeshi Watanabe, Tokushima-city (JP)

Correspondence Address: SUGHRUE MION, PLLC, 2100 PENNSYLVANIA AVENUE, N.W., SUITE 800, WASHINGTON, DC, 20037, US

(73) Assignee: OTSUKA PHARMACEUTICAL CO., LTD.

(21) Appl. No.: 10/787,098

(22) Filed: Feb. 27, 2004

Related U.S. Application Data
(60) Provisional application No. 60/465,214, filed on Apr. 25, 2003.

(51) Int. Cl. A01K 67/027; C07H 21/04; C12N 15/85; C12N 5/06

(52) U.S. Cl. 800/14; 435/353; 435/455; 530/350; 536/23.5

(57) ABSTRACT

Congenic rats are disclosed which express a truncated form of GPR10, wherein the truncation is the first 64 amino acids present in the NH₂-terminus of wild-type GPR10. Also disclosed are in vitro screening methods for identifying compounds that bind to or modulate the function or expression of the truncated GPR10, and thus are useful in treating psychiatric diseases, such as depression and anxiety.

### FIELD OF THE INVENTION

The present invention relates to congenic rats which express a truncated form of GPR10, wherein the truncation is the first 64 amino acids present in the NH₂-terminus of wild-type GPR10. The present invention also relates to in vitro screening methods for identifying compounds that bind to or modulate the function or expression of the truncated GPR10, and thus are useful in treating psychiatric diseases, such as depression and anxiety.

### BACKGROUND OF THE INVENTION

The Otsuka Long Evans Tokushima Fatty (OLETF) rat is a useful animal model for studying the mechanisms of obesity, dyslipidemia, and diabetes development (Diabetes, 41:1422 (1992)). Whole-genome quantitative trait loci (QTL) analyses has identified 12 statistically significant QTLs, termed Dmol through Dmol2, that are associated with obesity, dyslipidemia, and type II diabetes phenotypes in the OLETF rat. Dmol, located on the distal portion of the long arm of rat chromosome 1, has shown strong effects on body weight, visceral fat weight and plasma triacylglycerols levels (Mamm. Genome, 9:419 (1998); Genomics, 58:233 (1999); and Genomics, 62:350 (1999)).

Congenic lines for the Dmol locus have been established by introducing nondiabetic Dmol alleles from the Brown-Norway (BN) (Clin. Exp. Pharmacol. Physiol., 28:28 (2001)) and Fischer 344 (F344) rat strains (Genet. Res., 77:183 (2001)) into the OLETF rat background. The established congenic lines, which are heterozygous for the nondiabetic Dmol allele, showed significant improvements in obesity, dyslipidemia, hyper-insulinemia, hyperglycemia and glucose intolerance phenotypes in each generation.

Congenic lines, in which both OLETF Dmol alleles have been replaced with F344-derived alleles have also been produced (Diabetes Obes. Metab., 4:309 (2002); which is incorporated by reference herein in its entirety). These homozygous Dmol-F344/F344 congenic rats showed significant decreases in body weight, abdominal fat weight, serum triacylglycerols, total cholesterol, food consumption and blood glucose after glucose loading compared with Dmol-OLETF/OLETF animals.

In order to locate the genes that are important for obesity, dyslipidemia, hyper-insulinemia and hyperglycemia, in the present invention, a congenic rat was produced where the Dmol domain was placed in the OLETF background. After the area of domain was narrowed by observing changes in obesity and hyperlipemia, a mutant (NH₂-terminus truncated) GPR10, was discovered using positional cloning and the positional candidate approach.

GPR10 is a G-protein coupled receptor identified by screening a genomic DNA library. (Genomics, 29:335 (1995)); and prolactin-releasing peptide (PrRP) is an endogenous ligand for GPR10 (Nature, 393:272 (1998)). GPR10 is expressed abundantly in the hypothalamus, anterior pituitary and adrenal medulla, whereas PrRP is expressed mainly in the hypothalamus, medulla oblongata and intestine (Endocrinol., 140:5736 (1999)). PrRP neurons are widely disseminated throughout the brain, such as the paraventricular nucleus (PVN), the supraoptic nucleus (SON). The distribution pattern suggests that PrRP is involved in a variety of brain functions (Neuroendocrinol., 71:262 (2000)). The administration of PrRP to rats was found to reduce food intake and body weight (Nat. Neurosci., 3:645 (2000)), increase plasma oxytocin (Neurosci. Lett., 276:193 (1999)) and ACTH (Neurosci. Lett., 285:234 (2000)), and promote wakefulness (Psychiatry Clin. Neurosci., 54:262 (2000)).

In the present invention, a congenic rat has been produced by introducing the mutant GPR10 (OLETF type) domain into the BN background. It was surprisingly discovered that the mutant GPR10 congenic rats showed significant depression and anti-anxiety activities, but not obesity and hyperlipemia, compared with the normal BN rats.

### SUMMARY OF THE INVENTION

The present invention, inter alia, relates to Brown-Norway (BN) rats congenically expressing a mutant form of GPR10, wherein said mutation is a truncation of the first 64 amino acids present in the NH₂-terminus of wild-type GPR10. Biochemical, physiological and behavioral differences between rats from the same species expressing wild-type and truncated GPR10 are useful tools for identifying novel functional roles for GPR10 and the potential pathological relevance of GPR10 to human diseases. Thus, the present invention also relates to in vitro screening methods for identifying compounds that bind to or modulate the activity of the mutant GPR10. Such compounds are believed to be useful for treating diseases of the central nervous system (CNS), such as, but not limited to, depression, anxiety and schizophrenia. These screening methods are based the discovery in the present invention that PrRP binds to two high affinity domains within the first 64 amino acids of the NH₂-terminus of wild-type GPR10, and that this binding site is critical for the agonistic activity of PrRP at GPR10, as evidenced by the absence of PrRP binding and function in cells expressing cloned truncated GPR10. In the present invention, the physiological relevance of the NH₂-terminus has been demonstrated by comparing rats expressing wild-type and mutant forms of GPR10, the latter of which was found to display depressed-like and less anxious-like behaviors in animal models commonly used to predict antidepressant and anxiolytic drug activity.

### BRIEF DESCRIPTION OF THE DRAWINGS

FIG. 1 shows the nucleic acid sequence encoding rat wild-type GPR10 (SEQ ID NO:1).

FIG. 2 shows nucleic acid sequence encoding rat mutant GPR10 (SEQ ID NO:2), which was isolated from OLETF rats.

FIG. 3 shows the nucleic acid sequence encoding the NH₂-terminus fragment of human GPR10 (SEQ ID NO:3).

FIG. 4 shows the nucleic acid sequence of human wild-type GPR10 (SEQ ID NO:4).

FIG. 5 shows the nucleic acid sequence of human mutant GPR10 (SEQ ID NO:5).

FIG. 6 shows the amino acid sequence of rat wild-type GPR10 (370 amino acids) (SEQ ID NO:6) (Accession No. S77867).

FIG. 7 shows the amino acid sequence of rat mutant GPR10, which was isolated from OLETF rats (306 amino acids) (SEQ ID NO:7).

FIG. 8 shows the amino acid sequence of human wild-type GPR10 (370 amino acids) (SEQ ID NO:8) (Accession No. AB015745).

FIG. 9 shows the amino acid sequence of human mutant GPR10 (306 amino acids) (SEQ ID NO:9).

FIG. 10 shows the amino acid sequence of the N-terminus fragment of human GPR10 (SEQ ID NO:10).

FIG. 11 shows the structures of the rat and human GPR10 expression vectors used in the examples.

FIG. 12 shows a scheme for the preparation of the GPR10-mutant congenic rats.

FIG. 13 shows the results of a forced swimming test using congenic rats expressing mutant GPR10. **p<0.01 by two tailed t-test.

FIG. 14 shows [¹²⁵I]PrRP binding to CHO cells stably expressing cloned human wild-type GPR10. All data points are means of quadruplicate determinations from a single representative experiment.

FIG. 15 shows [¹²⁵I]PrRP binding to CHO cells stably expressing cloned human NH₂-terminus truncated GPR10. All data points are means of quadruplicate determinations from a single representative experiment.

FIG. 16 shows PrRP displacement of 0.05 nM [¹²⁵I]PrRP binding to CHO cells stably expressing cloned human wild-type GPR10. All data points are means of quadruplicate determinations from a single representative experiment.

FIG. 17 shows Compound X displacement of 0.05 nM [¹²⁵I]PrRP binding to CHO cells stably expressing cloned human wild-type GPR10. All data points are means of quadruplicate determinations from a single representative experiment.

FIG. 18 shows the effect of PrRP and Compound X on [³H]arachidonic acid release from CHO cells stably expressing cloned human wild-type GPR10. All data points are means of triplicate determinations from a single representative experiment.

FIG. 19 shows the effects of PrRP and Compound X on [³H]arachidonic acid release from CHO cells stably expressing cloned human NH₂-terminus truncated GPR10. All data points are means of triplicate determinations from a single representative experiment.

FIG. 20 shows the synergistic effect of Compound X on PrRP-stimulated [³H]arachidonic acid release from CHO cells stably expressing cloned human wild-type GPR10. *A significant treatment difference (P<0.0001) was detected between Compound X and the cocktail of Compound X+1.0 μM PrRP (unpaired t-test).

FIG. 21 shows the effects of Compound X on PrRP-stimulated [³H]arachidonic acid release from CHO cells stably expressing cloned human NH₂-terminus truncated GPR10. All data points are means of triplicate determinations from a single representative experiment. *A significant treatment difference (P<0.0001) was detected between Compound X and the cocktail of Compound X+1.0 μM PrRP (unpaired t-test).

### DETAILED DESCRIPTION OF THE INVENTION

In one embodiment, the present invention relates to congenic rats which produce a GPR10 mutant that is 64 amino acids shorter from N-terminal than wild-type GPR10. That is, a congenic rat comprising a mutant GPR10 gene, wherein said congenic rat is obtained by crossing a Otsuka Long-Evans Tokushima Fatty (OTELF) rat (ATCC No. 72016) with a wild-type rat, and wherein said congenic rat exhibits a prolonged immobilization time when assayed in a forced swimming test compared to said wild-type rat and anti-anxiety behavior in an elevated plus-maze test compared to said wild-type rat.

OTELF rat embryos used to obtain the congenic rats are described in U.S. Pat. No. 5,789,652 (which is incorporated by reference herein in its entirety), and were deposited with the American Type Culture Collection under ATCC No. 72016.

The particular wild-type rat employed is not critical to the present invention. For example, the wild-type rat may be a BN rat or F344 rat.

Whole-gene scans have identified Dmol as a major quantitative trait locus for dyslipidaemia and obesity in OLETF rat (Genomics, 58:233-239 (1999)).

Congenic rats in which BN-derived Dmol genome region is transferred onto the OLETF genetic background have revealed substantial therapeutic effects against obesity, dyslipidaemia and diabetes of the OLETF rat (Clin. Exp. Pharmacol. Physiol., 28:28-42 (2001)). Positional cloning and positional candidate approach of the Dmol region have, in the present invention, identified a mutation in the GPR10 coding sequences. To search the novel biological function of the mutant GPR10, in the present invention, another congenic rat was produced. OLETF-derived genetic intervals were transferred onto the BN genetic background by 4 successive backcrosses (N5) followed by intercrosses between heterozygous animals to establish homozygous congenic rat. This congenic rat did not present the extreme abnormal phenotype seen in the OLETF rat such as obesity, hyperlipidaemia and diabetes. Thus, this congenic rat is believed to be useful in behavioral tests, because of the avoidance of the body weight difference against the control rat. The GPR10 mutation site was detected so as to select animals useful in the behavioral test.

The mutant GPR10 gene consists essentially of the DNA sequence of SEQ ID NO:2.

The wild-type rat comprises a GPR10 gene having the DNA sequence of SEQ ID NO:1.

The OTELF rat comprises said mutant GPR10 gene.

The present invention also relates to a tissue or cell obtained from the congenic rat, wherein said tissue and cell express said mutant GPR10 gene, as well as a culture of the cell.

Furthermore, the present invention relates to an isolated DNA molecule encoding a mutant GPR10 protein consisting essentially of the amino acid sequence of SEQ ID NO:7 or SEQ ID NO:9, where preferably the isolated DNA molecule consists essentially of the DNA sequence of SEQ ID NO:2 or SEQ ID NO:5, respectively.

In addition, the present invention relates to an isolated fragment of mutant GPR10 consisting essentially of the amino acid sequence of SEQ ID NO:10.

The term “gene” is used herein to mean a double-stranded DNA and its constituent single-stranded DNA, whether sense or anti-sense, without regard to its length. Therefore, unless otherwise indicated, the gene (DNA) of the invention includes a double-stranded DNA containing a genomic DNA, a single-stranded DNA (sense strand) inclusive of the cDNA, a single-stranded DNA (anti-sense strand) having a sequence complementary to said sense strand, and fragments of said DNAs.

The gene (DNA) of the invention may contain a leader sequence, a coding region, exons and introns. The polynucleotide includes both RNA and DNA. The DNA includes cDNA, genomic DNA and synthetic DNA. The protein includes its fragments.

The gene of the invention can be easily produced and isolated by the general genetic engineering technology based on the sequence information on any specific example of the gene of the invention as disclosed herein (e.g., Molecular Cloning 2d Ed, Cold Spring Harbor Lab. Press (1989); Zoku Seikagaku Jikken Koza (Experiments in Biochemistry, Second Series): “Idenshi Kenkyuho (Methods in Gene Research) I, II, III”, The Biochemical Society of Japan (ed.), (1986)).

More particularly, such can be obtained by preparing a cDNA library from a suitable source, in which the gene of the invention can be expressed, by a routine procedure and selecting a desired clone from this library using a suitable probe or antibody specific to the gene of the invention (Proc. Natl. Acad. Sci., USA., 78:6613 (1981); and Science, 222:778 (1983)).

The source of cDNA, which can be used in the above procedure includes various cells and tissues expressing the gene of the invention, as well as cultured cells derived therefrom, particularly brain tissues. Isolation of the total RNA from such a source, isolation and purification of mRNA, and acquisition and cloning of cDNA can also be carried out in the conventional manner. Moreover, cDNA libraries are commercially available and the present invention can be carried into practice using such cDNA libraries, for example those cDNA libraries available from CLONTECH Lab. Inc.

The method of screening for the gene of the invention from a cDNA library is not particularly restricted but the conventional procedure can be employed.

Examples of the screening methods include an immunoscreening method using a specific antibody to the protein produced by a cDNA to select the corresponding cDNA clone, a method using a probe selectively binding to the objective DNA sequence, such as a plaque hybridization method, and a colony hybridization method, and a combination of such methods.

As the probe for the above method, the DNA chemically synthesized according to nucleotide sequence information on the gene of the invention can be generally employed. The gene of the invention which has already been obtained or a fragment thereof can also be used as a probe. A sense primer and anti-sense primer established according to the nucleotide sequence information on the gene of the invention can be used as screening probes.

The nucleotide sequence for use as the probe may be a partial nucleotide sequence comprising at least 10 consecutive nucleotides, preferably 20 consecutive nucleotides, more preferably 30 consecutive nucleotides, most preferably 50 consecutive nucleotides.

In obtaining the gene of the invention, DNA/RNA amplification by PCR (Science, 230:1350 (1985)) can be used. Particularly when a full-length cDNA can hardly be obtained from a library, the RACE method (Rapid Amplification of cDNA ends; Jikken Igaku (Experimental Medicine, 12(6):35 (1994))), especially 5′-RACE method (Proc. Natl. Acad. Sci., USA., 8:8998 (1988)), can be used.

The primers for use in such PCR methods can be judiciously established with reference to the sequence information on the gene of the invention as disclosed herein and can be synthesized by routine procedures. The isolation and purification of the amplified DNA/RNA fragment can be carried out in a routine manner as mentioned above, for example by the gel electrophoresis method.

For example, the mutation in GPR10 can be detected by PCR using reaction mixtures prepared form reagents provided in the PE Applied Biosystems TaqMan PCR Reagent Kit according to the manufacture's directions. For example, TaqMan probe 1 (CAGTGAGGTCATGGC (SEQ ID NO:11)) and probe 2 (CAGTGAGGTTATGGC (SEQ ID NO:12)), and forward primer GR-F1 (CCAGGAGAGAAGTTGGGTGA (SEQ ID NO:13)) and reverse primer GR-R1 (GACGGCCCAGAAAACAAAT (SEQ ID NO:14)) can be used to detect the rat GPR10 mutation. Total reaction volumes are 25 μl. The master reaction mix can be dispensed into optical PCR tubes in a volume of 24 μl. Rat genomic DNAs (1.0 μl) are then added to the reaction mix (below) and mixed thoroughly. PCR thermal cycling program consists of an initial cycle of 95° C. for 10 min followed by 40 cycles of 95° C. for 15 sec and 62° C. for 1 min. Reaction plates are loaded into a PE Applied Biosystems 7700 Sequence Detector and the nucleotide sequence of the GPR10 mutation site is analyzed.

| PCR mixture | | |
|---|---|---|
| Probe 1 (0.4 pmol/μl) | 2.5 | μl |
| Probe 2 (0.4 pmol/μl) | 2.5 | μl |
| Primer mix (3.0 μM each) | 2.5 | μl |
| Univ. Mix (2×) | 3.125 | μl |
| 2 × RB | 9.375 | μl |
| genome | 1.0 | μl |
| dH₂O | 4.0 | μl |
| Total | 25 | μl |

Sequencing of the gene of the invention as obtained in the above manner or various DNA fragments can be made in accordance with the dideoxy method (Proc. Natl. Acad. Sci., USA., 74:5463 (1977)) or the Maxam and Gilbert method (Methods in Enzymology, 65:499 (1980)) or more expediently by using a commercial sequencing kit.

For example, with the gene of the invention thus obtained, the expression or non-expression of the gene of the invention in an individual or a given tissue can be specifically detected by utilizing a portion or the entire nucleotide sequence of the gene of the invention.

The above detection can be made by conventional procedures, such as RNA amplification by RT-PCR (reverse transcribed-polymerase chain reaction; Kawasaki et al, “Amplification of RNA”, In PCR Protocol: A Guide to Methods and Applications, Academic Press, Inc., San Diego, pages 21-27 (1991)), Northern blot analysis (Molecular Cloning, Cold Spring Harbor Lab. (1989)), determination on cellular level by in situ RT-PCR (Nucl. Acids Res., 21:3159-3166 (1993)) or in situ hybridization, NASBA (nucleic acid sequence-based amplification, Nature, 350:91-92 (1991)), and the like conventional techniques. The preferred technique is the RT-PCR detection method.

The primer which is to be used when the PCR method is chosen for the above purpose is not particularly restricted insofar as it is characteristic of the gene of the invention and capable of selective amplification of the particular gene only and can be judiciously established based on the sequence information on the gene of the invention. Usually, one having a partial sequence of the gene of the invention, which is about 10-35 nucleotides long, preferably about 15-30 nucleotides long can be used as the primer.

The gene of the invention, thus, includes the DNA fragment which can be used as a specific primer and/or specific probe for the detection of the gene of the invention.

The DNA fragment mentioned above can be defined as a polynucleotide which hybridizes with the polynucleotide under stringent conditions. The stringent conditions mentioned above may be the ordinary conditions for primers or probes and, as such, are not particularly restricted. For example, as the above-mentioned conditions of 6×SSC, 65° C., overnight or the condition of 50% formamide and 4×SSC, 37° C., overnight can be employed.

By applying the gene of the invention to standard genetic engineering technology, the expression product (polypeptide) of the gene or a protein containing it can be easily produced in large quantities and with good reproducibility.

Therefore, the invention further provides a polypeptide having the amino acid sequence encoded by the gene of the invention (the expression product of the invention), a vector harboring the gene of the invention for the production of the polypeptide, a host cell transfected with the vector, and a method of producing the polypeptide of the invention which comprises growing the host cell.

The polypeptide (GPR10 protein) having the amino acid sequence shown is a specific embodiment of the polypeptide of the invention.

The polypeptide of the invention can be produced by the conventional recombinant DNA technology (e.g., Science, 224:1431 (1984); Biochem. Biophys. Res. Comm., 130:692 (1985); Proc. Natl. Acad. Sci., USA., 80:5990 (1983)) based on the gene sequence information provided by the present invention.

More particularly, the production of said polypeptide is carried out by the procedure comprising constructing a recombinant DNA (expression vector) which permits expression of the gene coding for the desired protein in a host cell, transforming the host cell with the vector, growing the resulting transformant, and harvesting the polypeptide from the culture broth.

The host cell mentioned above may be a prokaryotic cell or an eukaryotic cell. As the prokaryotic host, Escherichia coli, Bacillus subtilits and other common bacteria can be employed and preferably cells of Escherichia coli, particularly cells of Escherichia coli K12, can be employed. The eukaryotic host cell includes cells of vertebrates and yeasts and the former include the monkey cell line COS (Cell, 23:175 (1981)), Chinese hamster ovarian cells, and the dihydrofolate reductase-defective cells thereof (Proc. Natl. Acad. Sci., USA., 77:4216 (1980)). As the latter, yeast cells of the genus Saccharomyces can be used, but these are not exclusive choices.

When prokaryotic cells are used as host cells, an expression plasmid construct prepared by using a vector which is replicable in the particular host cell and adding a promoter and SD (Shine and Dalgarno) sequence upstream of the gene of the invention so that the gene may be expressed therein as well as an initiation codon (e.g., ATG) necessary for initiation of protein synthesis can be used. As the vector mentioned above, it is usual to employ plasmids derived from Escherichia coli, such as pBR322, pBR325, pUC12, pUC13, etc. However, these are not exclusive choices but various known vectors can be utilized. Examples of the commercial vectors for use in expression systems using E. coli include pGEX-4T (Amersham Pharmacia Biotech), pMAL-C2, pMAl-P2 (New England Biolabs), pET21, pET21/lacq (Invitrogen) and pBAD/His (Invitrogen).

As the expression vector for use when cells of a vertebrate are used as host cells, the vector having a promoter upstream of the gene of the invention to be expressed, RNA splice sites, polyadenylation site and a transcription termination sequence is usually employed, and this vector may further have a replication origin where necessary. A specific example of the expression vector is pSV2dhfr harboring an early promoter of SV40 (Mol. Cell. Biol., 1:854 (1981)). Aside from the above, various known vectors available commercially can be employed. Examples of the commercial vectors which are used in expression systems using animal cells include vectors for animal cells, such as pEGFP-N, pEGFP-C (CLONTECH), pIND (Invitrogen), pcDNA3.1/His (Invitrogen), etc., and vectors for insect cells, such as pFastBac HT (Gibci BRL), pAcGHLT (PharMingen), pAc5/V5-His, pMT/V5-His and pMT/Bip/V5-his (all Invitrogen).

pAM82 having a promoter for the acid phosphatase gene (Proc. Natl. Acad. Sci., USA., 80:1 (1983)) is a specific example of the expression vector for use when yeast cells are used as host cells. The commercial expression vectors for yeast cells include pPICZ (invitrogen) and pPICZα (Invitrogen).

The promoter is not particularly restricted, either. When a strain of the genus Escherichia is used as the host, tryptophan (trp) promoter, lpp promoter, lac promoter, recA promoter, PL/PR promoter, etc. can be utilized. When the host is a strain of the genus Bacillus, SP01 promoter, SP02 promoter, penP promoter, etc. are preferably used. When a yeast is used as the host, pH05 promoter, PGK promoter, GAP promoter, ADH promoter, etc. can be utilized. The preferred promoter for use when host cells are animal cells include SV40-derived promoters, retrovirus promoters, metallothionein promoter, heat shock promoter, cytomegalovirus promoter, and SRα promoter.

As the expression vector for the gene of the invention, a conventional fusion protein expression vector can be used. PGEX (Promega) for the expression of glutathione-S-transferase (GST)-fused proteins is a specific example of the vector.

The polynucleotide sequence wherein the coding sequence for a mature polypeptide assists in the expression and secretion of a polypeptide from host cells includes the secretory sequence, the leader sequence and the marker sequence (hexahistidine tag, histidin tag) used in the purification of a fusion mature polypeptide in the case of bacterial cells, and the hemaglutinin (HA) tag in the case of mammalian cells.

By example, the invention also relates to expression vectors comprising a nucleic acid encoding rat and human GPR10 and mutant GPR10 as described in Examples 1-2 below.

The method of introducing the recombinant DNA (expression vector) into the host cell and the associated transforming method are not particularly restricted, and various standardized methods can be utilized.

The transformant obtained can be cultured in the routine manner, whereby the objective protein encoded by the deliberately designed gene according to the invention is expressed and produced (accumulated/secreted) intracellularly, extracellularly or on the cell membrane.

The culture medium to be used can be judiciously selected from among various routine media according to the kind of adopted host cell and the culture is also performed under conditions favoring growth of the host cell.

The resulting recombinant protein (GPR10 protein) according to the invention can be optionally isolated and purified by various separation techniques taking advantage of its physical and/or chemical properties, for instance (“Seikagaku Data Book (Biochemical Data Book) II”, pages 1175-1259, First Edition, 1st impression, Tokyo Kagaku Dojin K. K. (Jun. 23, 1980); Biochemistry, 25(25):8274 (1986); Eur. J. Biochem., 163:313 (1987), etc.).

Examples of such techniques are the conventional reconstitution method, treatment with a protein precipitating agent (salting-out method), centrifugation, osmotic shock method, sonic disruption, ultrafiltration, various types of chromatography such as molecular sieve chromatography (gel filtration), adsorption chromatography, ion exchange chromatography, affinity chromatography and high performance liquid chromatography (HPLC), dialysis, and combinations of these techniques. The particularly preferred technique includes affinity chromatography using a column to which a specific antibody to the protein of the invention has been coupled.

In designing the objective gene encoding the polypeptide of the invention, the nucleotide sequence of the gene can be utilized. If desired, this gene can be used after the codons specifying the respective amino acid residues have been judiciously altered. Furthermore, when any amino acid residue or partial sequence of the amino acid sequence encoded by the gene is to be modified by substitution, deletion or addition, such modifications can be made by the various methods described above, for example by site-specific mutagenesis.

The polypeptide of the invention can be produced by the standard protocol for chemical synthesis according to the amino acid sequence shown herein. The method includes the conventional liquid-phase method and solid-phase method for peptide synthesis.

More particularly, the method for peptide synthesis includes the so-called stepwise elongation method in which the constituent amino acids are coupled one by one for chain extension and the fragment condensation method which comprises synthesizing fragments each consisting of several amino acids beforehand and coupling the fragments together. The synthesis of the protein of the invention can be carried out by whichever of the above two methods.

The method of condensation for use in the above peptide synthesis may also be a conventional ones including the azide process, mixed acid anhydride process, DCC process, active ester process, redox process, DPPA (diphenylphosphoryl azide) process, DCC+additive (1-hydroxybenzotriazole, N-hydroxy-succinamide, N-hydroxy-5-norbornene-2,3-dicarboximide or the like) process and Woodward's reagent process.

The solvent to be used in these processes can also be judiciously selected from among the common solvents well-known in the art for use in such peptide-forming condensation reactions. Examples of the solvents include dimethylformamide (DMF), dimethyl sulfoxide (DMSO), hexaphosphoramide, dioxane, tetrahydrofuran (THF), ethyl acetate, etc., and mixtures thereof.

In conducting the peptide synthesizing reactions, the carboxyl group of any amino acid or fragment peptide that should not take part in the reaction can be protected in advance, generally by esterification in the form of a lower alkyl ester such as methyl ester, ethyl ester, tert-butyl ester, etc., or an aralkyl ester such as benzyl ester, p-methoxybenzyl ester, p-nitrobenzyl ester, etc.

Referring to any amino acid having a functional group in its side chain, the hydroxyl group of a tyrosine residue, for instance, may be protected in advance with an acetyl, benzyl, benzyloxycarbonyl, tertiary butyl or other group, although such protection is not necessarily indispensable. Furthermore, the guanidino group of an arginine residue can be protected with a suitable protective group such as nitro, tosyl, p-methoxy-benzene-sulfonyl, methylene-2-sulfonyl, benzyloxycarbonyl, isobornyloxycarbonyl, adamantyloxycarboxyl or the like.

The reactions for eliminating such protective groups from the protected amino acids, peptides or the end product protein of the invention can also be carried out in the routine manner, for example by catalytic reduction or a method using liquid ammonia/sodium, hydrogen fluoride, hydrogen bromide, hydrogen chloride, trifluoroacetic acid, acetic acid, formic acid, methane-sulfonic acid or other reagent.

The polypeptide of the invention, thus produced, can be purified as needed by the various techniques mentioned above, such as ion exchange resin chromatography, partition chromatography, gel chromatography, countercurrent distribution and the like methods in routine us in the field of peptide chemistry.

The polypeptide of the invention can be used as an immunogen for preparation of its specific antibody. By utilizing this immunogen, the antiserum (polyclonal antibody) and the monoclonal antibody can be provided.

The technology of producing antibodies is well-known to those skilled in the art and the known procedures can be employed in the present invention (e.g. Zoku Seikagaku Jikken Koza (Experiments in Biochemistry, second series) “Men-eki Seikagaku Kenkyuho (Methods in Immunobiochemistry)”, edited by the Biochemical Society of Japan (1986)).

For example, as the immune animal for harvesting the desired antiserum therefrom, the ordinary animals such as rabbit, guinea pig, rat, mouse, chicken, etc., can be arbitrarily selected and the immunization with said immunogen and the collection of blood can also be carried out by the conventional procedures.

Preparation of a monoclonal antibody can also be carried out by the conventional technique which comprises constructing a hybridoma between the plasma cell (immune cell) of an animal immunized with said immunogen and a plasmacytoma cell, selecting clones producing the desired antibody, and cultivating the clones. The immune animal is generally selected in consideration of its compatibility with the plasmacytoma cell to be used for cell fusion and usually the mouse or the rat is used. The immunization procedure may be the same as used for the preparation of said antiserum and, if desired, the immunization can be made using a conventional adjuvant in combination.

The plasmacytoma cell for use in said hybridization is not particularly restricted, either, but includes various myeloma cells such as p3 (p3/x63-Ag8) (Nature, 256:495-497 (1975)), p3-U1 (Current Topics in Microbiology and Immunology, 81:1-7 (1978)], NS-1 (Eur. J. Immunol., 6:511-519 (1976)), MPC-11 (Cell, 8:405-415 (1976)), SP2/0 (Nature, 276:269-271 (1978)), etc., R210 (Nature, 277:131-133 (1979)) and others in rats, and cells derived therefrom.

The hybridization between said immune cell and said plasmacytoma cell can be effected by the known technology in the presence of a conventional hybridization promoter such as polyethylene glycol (PEG) or Sendai virus (HVJ) and the separation of the objective hybridoma can also be carried out in the known manner (Meth. in Enzymol., 73:3 (1981); Zoku Seikagaku Jikken Koza (ditto)).

The search for the objective antibody-producing cell clone and the monoclonal antibody preparation can also be carried out in the routine manner. For example, the search for the antibody-producing hybridoma can be made by any of the various techniques in routine use for the detection of antibodies, such as ELISA (Meth. in Enzymol., 70:419-439 (1980)), plaque method, spot method, agglutination reaction method, Ouchterlony method, radioimmunoassay, and the like, using the protein of the invention as an antigen.

Harvesting of the antibody of the invention from the resulting hybridoma can be achieved by cultivating the hybridoma in the routine manner and recovering the antibody as a culture supernatant or administering the hybridoma to a compatible mammal and recovering the antibody in the form of ascites. The former method is suitable for production of the antibody of high purity, while the latter method is suitable for high-production of the antibody. The antibody thus produced can be further purified by the conventional means such as salting-out, gel filtration, affinity chromatography and the like.

The antibody thus obtained is characterized by its binding affinity for the GPR10 protein of the invention and can be used for the purification of GPR10 protein and determination or differentiation of the protein by immunological techniques. Furthermore, this antibody can be utilized in the screening for agonists or antagonists of GPR10 protein.

The present invention provides the novel antibody described above, too.

Furthermore, in accordance with the invention, for the purpose of detecting the presence of the gene, it is possible to prepare a biological sample such as blood or serum, optionally extract the nucleic acid, and analyzing it for the gene.

The method of detecting the gene may comprise preparing a DNA fragment of the gene of the invention and design it so that it may be used in the screening for the gene and/or its amplification. More specifically, it is possible to construct a DNA fragment having the properties of a probe for plaque hybridization, colony hybridization, Southern blotting, Northern blotting, etc. or a probe for the preparation of a full-length or partial DNA of the gene of the invention as amplified by a polymerase chain reaction (PCR) which amplifies a nucleotide sequence with a polymerase. For this purpose, a primer having the same sequence as the gene is first prepared. Then, this primer is reacted, as a probe for screening, with a biological sample (nucleic acid sample) to check for the presence of the particular the gene sequence. The nucleic acid sample may be prepared by any of various techniques facilitating detection of the target sequence, such as denaturation, restriction enzyme digestion, electrophoresis or dot blotting.

As the method for said screening, the use of a PCR technique is particularly preferred from sensitivity points of view, and this technique is not particularly restricted inasmuch as a fragment of the gene of the invention is used as a primer. Thus, it can be utilized that any of the hitherto-known techniques (Science, 230:1350-1354 (1985)) and the modified versions of PCR which have been developed of late or will be developed in the future (Sakaki Yoshiyuki et al (ed.), Jikken Igaku (Experimental Medicine), Supplement 8(9) (1990), Yodosha; and Protein, Nucleic Acid, Enzyme: Special Supplement, Kyoritsu Shuppan, 35(7) (1990)).

The DNA fragment for use as the primer is a chemically synthesized oligo-DNA, and such oligo-DNA can be synthesized using an automated DNA synthesizer or the like, for example Pharmacia LKB Gene Assembler Plus (Pharmacia). The preferred length of the primer (sense primer or anti-sense primer) to be synthesized may for example be about 10-30 nucleotides. The probe for use in said screening is usually a labeled probe but may be an unlabeled one, or the detection may be made according to specific binding to a directly or indirectly labeled ligand. The suitable label and the method of labeling the probe or ligand belong to the prior art. Thus, the prior art label includes radioisotopes, biotin, fluorescent groups, chemiluminescent groups, enzymes, antibodies, etc., which can be taken up through known procedures such as nick translation, random priming and kinase treatment.

The PCR technique to be used for detection may for example be RT-PCR but various modifications of the technique which are in routine use in the art can be utilized.

Furthermore, the above assay method can be expediently carried out by utilizing an reagent kit for detecting the gene in samples.

Therefore, the present invention provides an gene detection reagent kit comprising a DNA fragment of the gene of the invention.

This reagent kit comprises at least a DNA fragment which hybridizes with a part or the entire nucleotide sequence or its complementary nucleotide sequence as an essential component and may optionally contain other components such as a labeling agent and PCR reagents (for example, Taq DNA polymerase, deoxynucleotide triphosphates, primers, etc.).

The labeling agent may be a radioisotope or a chemical modifier such as a fluorescent substance but the DNA fragment as such may have been conjugated with such a labeling agent. This reagent kit may further contain a suitable reaction solvent or diluent, standard antibody, buffer, wash solution, reaction stopper solution, etc. which make an assay easier to perform.

By the direct or indirect sequencing of the genes obtained from test samples by utilizing the above method, it is possible to find new gene-related genes having high homology to the wild-type gene.

Therefore, the present invention further provides a method of screening for GPR10 gene-related genes in samples which comprises performing said assay and sequencing of the genes contained in test samples.

The wild-type GPR10 and/or mutant GPR10 can be determined by utilizing the protein encoded by the GPR10 gene of the invention, a polypeptide having an amino acid sequence derived from the sequence by the deletion, substitution or addition of 1 or a plurality of amino acids, a fragment of either of them, or an antibody to any of such proteins.

Therefore, the invention provides a method of determining an anti-wild-type GPR10 and/or mutant GPR10 antibody or a method of determining the antigen. By this method, the degree of impairment of the brain nerve can be detected from a change in wild-type GPR10 (polypeptide). Such changes can be detected by the sequencing of GPR10 by the well-established technology described hereinabove, more preferably by detecting differences in the GPR10 polypeptide or the presence or absence of GPR10 polypeptide by the use of said antibody (polyclonal or monoclonal antibody).

The following is a specific example of determination of said wild-type and/or mutant GPR10. The anti-GR10 antibody can be used to immunoprecipitate GPR10 polypeptide from a solution containing a biological sample obtained from a body, such as blood or serum or can be reacted with the GPR10 polypeptide on polyacrylamide gel of Western blot or immunoblot. The GPR10 polypeptide in a paraffin section or frozen tissue specimen can be detected by an immunohistochemical technique using the anti-GPR10 antibody. The antibody production and purification technology are well-known in the art and suitable techniques can be selectively employed.

The preferred technology relevant to the detection of a wild-type GPR10 or a mutant thereof includes enzyme-linked immunosorbent assay (ELISA), radioimmunoassay (RIA), immunoradiometric assay (IRMA) and immunoenzymometric assay (IEMA) with a sandwich technique using a monoclonal antibody and/or a polyclonal antibody.

As noted above, the invention also relates to vectors (e.g., expression vectors) comprising a nucleic acid encoding rat and human GPR10 and mutant GPR10, e.g., the vectors as described in Example 1-2 below.

By comparing the congenic rats of the present invention (or their tissues or cells) to wild-type rats (or their tissues or cells), it is possible to screen for the biological functions of GPR10, and to screen for compounds that inhibit GPR10 protein activity by, e.g.,:

(a) contacting a test compound with:

(i) the congenic rat of claim 1, and

(ii) the wild-type rat of claim 1; and

(b) assaying for GPR10 activity in the resulting rats (i) and (ii),

wherein when said activity is found to differ in said rats (i) and (ii), said test compound is identified as a compound that inhibits GPR10 protein activity.

GPR10 activity is preferably depression in a forced swimming test or fear and anxiety in an elevated plus-maze test; or by:

(a) contacting a test compound with:

(i) the tissue or cell expressing mutant GPR10, or the isolated mutant GPR10 protein, and with

(ii) the tissue or cell obtained from a wild-type rat which expresses wild-type GPR10 protein or wild-type GPR10 protein, respectively; and

(b) assaying for GPR10 activity in the resulting tissues or cells or isolated proteins (i) and (ii),

wherein when said activity is found to differ in said tissues or cells or isolated proteins (i) and (ii), respectively said test compound is identified as a compound that inhibits GPR10 protein activity.

An example of the above screening is a screening system in which prokaryotic or eukaryotic host cells transformed stably with a recombinant DNA coding for an the polypeptide, or a fragment thereof, are used in, preferably, a competitive binding assay, or release of arachidonic acid metabolite. More preferably, the GPR10 protein activity is binding of said test compound to GPR10 protein as determined using [¹²⁵I]-PrRP in a competition assay, or GPR10 activity is measured in a [³H]-arachidonic acid metabolite release. As an alternative, said host cells, whether in the free form or as immobilized, are used in the standard binding assay. More particularly, the above screening may comprise reacting the GPR10 polypeptide, or a fragment thereof and PrRP in the presence of a candidate drug, to cause formation of a complex and detecting the degree of inhibition of the complex formation by the above candidate drug.

Thus, in accordance with the invention, there can be provided a method for screening which comprises contacting a candidate drug with the GPR10 polypeptide, or a fragment thereof and, then, detecting the presence of the resulting complex or the presence of a complex of the GPR10 polypeptide, or a fragment thereof, with a ligand by a per se known technique. Furthermore, by assaying GPR10 activity, it is possible to evaluate whether a candidate drug is capable of antagonizing PrRP and accordingly may modify the above-defined GPR10 activity. In such a competitive binding assay, the GPR10, or a fragment thereof, is labeled. When the free GPR10 polypeptide or fragment thereof is separated from the protein-protein complex and the labeling amount of the free (non-complex-forming) substance is measured, the measured value serves as a yardstick of the binding of the test factor to the PrRP. The measured value serves also as a measure of inhibition of the binding of PrRP to the GPR10 polypeptide. By analyzing a small peptide (pseudopeptide) of the GPR10 polypeptide in this manner, the candidate drug can be assayed as a substance having PrRP antagonizing activity.

As a further method for drug screening, the GPR10 polypeptide of the invention or the GPR10 gene product of the invention can be used in the screening for compounds which activate (agonists) or inhibit (antagonists or inhibitors) the activity of the GPR10 polypeptide or GPR10 gene product.

By using the GPR10 polypeptide or GPR10 gene product of the invention, agonists or antagonists can be identified from cells, cell-free preparations, chemical libraries and naturally-occurring compositions. These agonists or antagonists may be natural or modified substrates, ligands, enzymes or receptors of the GPR10 polypeptide of the invention or structural or functional copies of the polypeptide of the invention (Coligan et al, Current Protocols in Immunology, 1(2), Chapter 5 (1991)).

An agonist or antagonist of the GPR10 protein or an GPR10 gene product is expected to find application as a therapeutic or prophylactic drug for diseases of the central nervous system (CNS), such as, but not limited to, depression, anxiety and schizophrenia.

Compounds obtainable by the screening for candidate drugs for said GPR10 gene-related diseases have the functions of the protein of the invention (the expression product of the gene of the invention), such as depression, anxiety and schizophrenia. Thus, the proteins of the invention (inclusive of the gene expression products, partial peptides thereof, and salts thereof) are of use as reagents for the screening for compounds which promote the functions of the protein of the invention.

The invention provides a method of screening for compounds which promote the functions of the protein of the invention (hereinafter each referred to sometimes as a functional enhancer of the protein of the invention). More particularly, the invention provides (a) a method of screening for a functional enhancer of the protein of the invention which comprises contacting (1) the protein of the invention with nerve cells or a nerve tissue on one hand and (2) the protein and a test compound with said nerve cells or tissue on the other hand and comparing the results and (b) a method of screening for a functional enhancer of the protein of the invention which comprises administering (1) the protein of the invention to a vertebrate on one hand and (2) the protein of the invention and a test compound to the vertebrate on the other hand and comparing the results.

More particularly, in the above screening method (a), a physiological activity in the central nervous systems, is measured under the above conditions (1) and (2) and the results are compared. In the screening method (b), the mnemonic (memory-forming) activity in the brain, for instance, is measured under said two conditions (1) and (2) and the results are compared.

The nerve cells (neurous and neuroglia) for use in the above screening include neuroblastoma cells, glioma cells, and their hybridoma cells (e.g., N18TG-2, IMR-32, GOTO (e.g., GOTO-P3), NB1, C6BU-1, U251, KNS42, KNS81 and NG108-15 cells, and PC12 cells having a potency of differentiation to nerve cells). The nerve tissue which can be used includes the mouse neuroepithelial cell, rat hippocampus primary culture cell, fetal mouse culture Prukinje cell, and mouse dorsal root ganglia. The test compound includes peptides, proteins, nonpeptide compounds, synthetic compounds, fermentation products, cell extracts, plant extracts, animal tissue extracts, and plasma. These compounds may be novel compounds or known compounds.

In carrying out said screening method (a), the protein of the invention (inclusive of a partial peptide thereof or a salt thereof) is dissolved or suspended in a screening buffer to prepare a sample of the protein of the invention. The buffer may be any buffer solution that does not interfere with the contact between the protein of the invention and the nerve cell or tissue (e.g., phosphate buffer, Tris-HCl buffer, etc. at pH about 4-10, preferably pH about 6-8). The duration of contact is usually about 1-10 days, preferably about 7-10 days. The contact temperature is usually about 37° C. The activities of the protein of the invention in the central nervous systems can be determined by the routine methods such as visual assessment of axonal elongation, measurement of intracellular Ca²⁺ concentration, and the like.

Any test compound promoting any of said physiological activities by at least about 20%, preferably not less than about 30%, more preferably not less than about 50%, still more preferably not less than about 70%, under the above-mentioned condition (2) as compared with the condition (1) can be selected as a functional enhancer of the protein of the invention.

Thus, the present invention also relates to a method for screening for a compound useful in treating depression or anxiety comprising:

(a) administering the compound that modulates GPR10 activity, to a mammal suffering from depression or anxiety; and

(b) assaying for amelioration of said depression or anxiety in the resulting mammal so as to identify a compound useful in treating depression or anxiety.

In carrying out the above screening method, the protein of the invention, alone or in combination with the test compound, is administered to test animals by intravenous, subcutaneous or intramuscular injection or orally. The dosage of the protein of the invention for oral administration is generally about 0.1-100 mg/day, preferably about 1.0-50 mg/day, more preferably about 1.0-20 mg/day, per mammal (based on 50 kg body weight). The parenteral dose should be selected according to the recipient and the method of administration but it is preferable to administer about 0.01-30 mg/day, preferably about 0.1-20 mg/day, more preferably about 0.1-10 mg/day, per mammal (50 kg body weight) by the intravenous route.

Test animals include such mammals as man, monkey, chimpanzee, mouse, rat, rabbit, sheep, swine, bovine, horse, cat and dog and fish (e.g. carp, salmon, herring, rainbow trout, goldfish, etc.).

The activity of the protein of the invention in the brain can be assayed in accordance with, for example, a water maze test protocol (Morris, J. Neurosci. Meth., 11:47-60 (1984)). Any test compound promoting the above mnemonic effect by not less than about 20%, preferably not less than 50%, more preferably not less than 70%, under said condition (2) as compared with said condition (1) is of use as a functional enhancer of the protein of the invention.

The screening kit as a further embodiment of the invention contains the protein of the invention (inclusive of the expression product of the gene, a partial peptide thereof, and any salt of either of them) as an essential component. A kit consists of the following components 1-4 is an example of the screening kit of the invention:

Component 1: Hanks solution as assay buffer,

Component 2: Protein standard (protein of the invention or a salt thereof),

Component 3: Nerve cells or a nerve tissue (a culture of said nerve cells or nerve tissue in a 24-well plate, 10⁴ cell/well, as grown using Eagle's MEM, Hanks solution under 5% CO₂ at 37° C.), and

Component 4: An inverted microscope for observation.

The screening with the above screening kit can be carried out as follows:

[Method]

The number per field of vision of axonal elongation-positive cells in the well containing the test compound is counted and compared with the number of axonal elongation-positive cells in the control (test compound-free) well and the difference is statistically tested.

The compound or salt obtained by the screening method or with the screening kit in accordance with the invention is a member selected from the above-mentioned class consisting of peptides, proteins, nonpeptide compounds, synthetic compounds, fermentation products, cell extracts, plant extracts, animal tissue extracts, etc. and is a compound capable of promoting the function of the protein of the invention. The compound that promotes the functions of the protein of the invention as such may show physiological activities which thereby promote the function of the protein of the invention or the like additively or synergistically or, although not showing such physiological activities by itself, may promote the function of the protein of the invention. Examples of the salts of the compound include salts with physiologically acceptable bases (e.g., alkali metals) or acids (e.g., organic acids, inorganic acids). Particularly preferred are physiologically acceptable acid addition salts, such as salts with inorganic acids (e.g., hydrochloric acid, phosphoric acid, hydrobromic acid, sulfuric acid) or organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid).

The compound or salt which promotes the function of the protein of the invention is of value as a safe, low-toxicity therapeutic-prophylactic drug for various diseases of the central nervous system as noted above.

The above screening procedure involves the use of cells which express the GPR10 polypeptide on the cell surface or respond to the protein of the invention. Among such cells are cells derived from mammalian animals, yeasts, Drosophilia and E. coli. The cells which express the GPR10 polypeptide (or the cell membrane having the expressed polypeptide) or respond to the GPR10 polypeptide is contacted with the test compound to observe the stimulation or inhibition of binding or functional response. Then, GPR10 activity of cells contacted with the candidate compound is compared with that of similar cells not contacted.

The above assay can be carried out by detecting adhesion to cells harboring the GPR10 polypeptide using a label directly or indirectly coupled to a candidate compound or in an assay system utilizing a competition with a label-competitive substance. In this manner, the binding of the candidate compound can be easily tested. Furthermore, using a detection system suited to cells bearing the GPR10 polypeptide in such assays, it may be tested whether the candidate compound will produce a signal ascribable to activation of the GPR10 polypeptide. The activation inhibitor is generally assayed in the presence of a known agonist and the effect of the candidate compound on the activation due to the agonist is observed. The assay may comprise a simple procedure comprising mixing the candidate compound with a solution containing the GPR10 polypeptide to form a mixture, determining the GPR10 activity in the mixture, and comparing the GPR10 activity of the mixture with a standard.

The low molecular compound (agonist or antagonist) which binds to the GPR10 protein can be obtained by a screening with BIACORE 2000, for instance (Markgren et al, Analytical Biochemistry, 265:340-350 (1998)).

In accordance with the invention, for the purpose of developing a more active or stabilized GPR10 polypeptide derivative or a drug which enhances or blocks the function of the GPR10 polypeptide in vivo, it is possible to construct a biologically active polypeptide or a structural analog thereof for interaction, such as an GPR10 agonist, GPR10 antagonist, GPR10 inhibitor or the like. The structural analog mentioned above can be obtained, for example, by determining the three-dimensional structure of a complex of GPR10 polypeptide with another protein by X-ray crystallography, computer modeling or a combination of such techniques. Information on the structure of a structural analog can also be acquired by polypeptide modeling based on the structures of homologous proteins.

To obtain said more active or stabilized GPR10 polypeptide derivative, analysis by alanine scan can be employed. This method comprises substituting Ala for each amino acid residue to assess the influence of substitution on peptide activity. Thus, as each amino acid residue of a peptide is thus analyzed, the region of importance to the activity or stability of the peptide is determined. By this method, it is possible to design a more active or stable GPR10 polypeptide derivative.

It is also possible to isolate the target-specific antibody selected by the functional assay and analyze its crystal structure. As a rule, by this approach, the pharmacore providing a basis for subsequent drug design is obtained. By producing an anti-ideotypic antibody to the functional pharmacologically active antibody, it is possible to identify and isolate a peptide from a chemically or biologically generated peptide bank. Therefore, it is predictable that the selected peptide may also serve as a pharmacore.

In this manner, it is possible to design and develop drugs having improved or stabilized GPR10 activity or acting as inhibitors, agonists or antagonists of GPR10 activity.

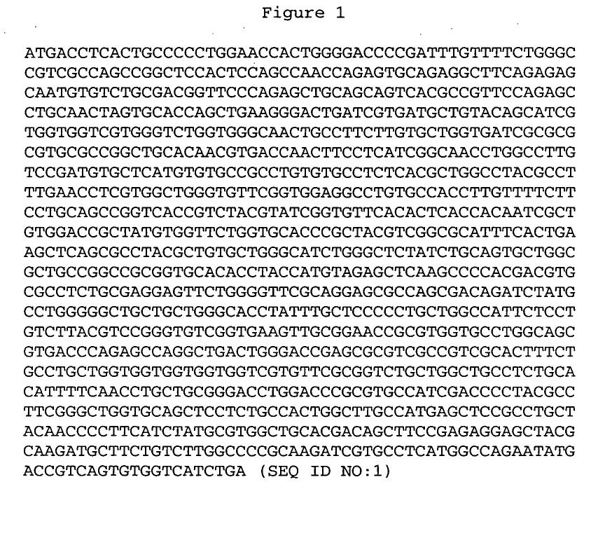

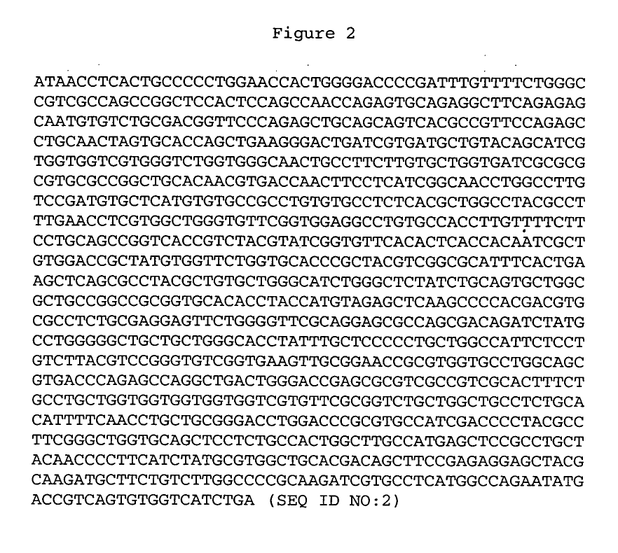

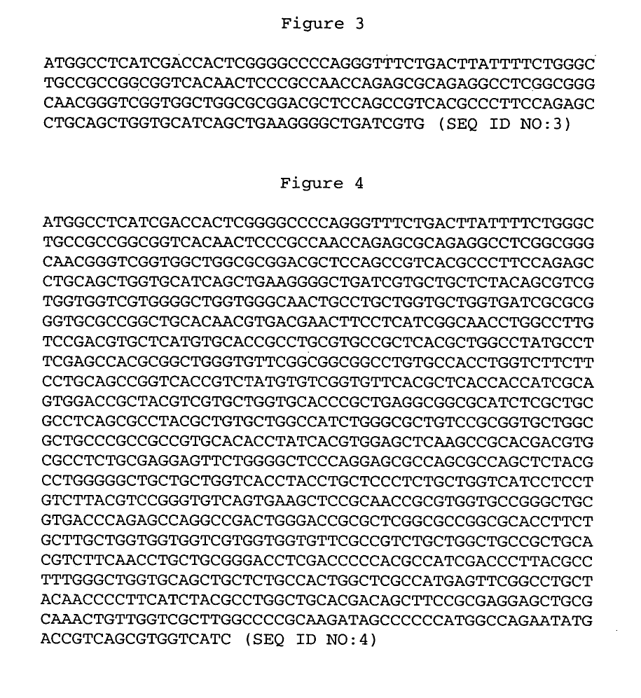

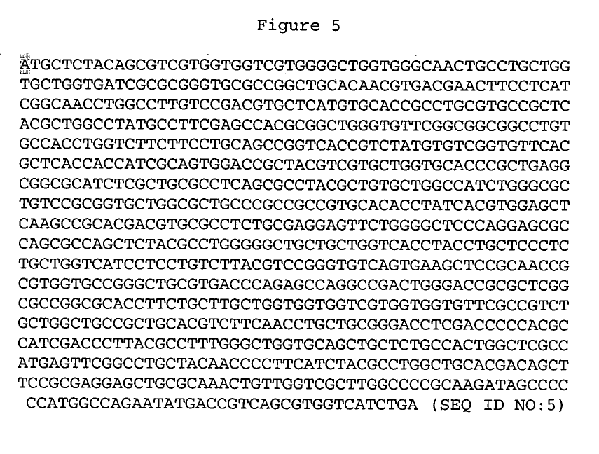

As discussed above, the invention provides screening methods for identifying compounds useful for the treatment of psychiatric diseases, such as, but not limited to depression and anxiety. In vitro methods are described which enable the identification of candidates or compounds (e.g., peptides, small molecules, antibodies, or the other drugs) that bind to and/or modulate wild-type GPR10 (see FIG. 4) and a truncated form of cloned human GPR10 (see FIG. 5) heterologously expressed in cells. These methods may also be applied as screening methods for use with other mammalian forms of GPR10, such as the wild-type and truncated forms of rat GPR10 (see FIGS. 1 and 2, respectively). In addition, these screening methods can be employed to calculate in vitro estimates of binding affinity, potency and relative intrinsic activity of cloned GPR10 receptors, by testing candidates at multiple concentrations and using statistical methods described in Examples 6-8 below, respectively.

### [¹²⁵I]PrRP Competition Binding Assay for Detecting Compounds that Bind to Cloned Wild-type GPR10

Chinese hamster ovary cell lines are generated that stably express cloned wild-type GPR10 receptors (pcNDA3.1(+)-hGPR10: CHO-hGPR10 cells) using the methods described in Examples 2-3 below. CHO-hGPR10 cells are cultured until approximately 70% confluent, washed three times with ice-cold homogenization buffer comprising 20 mM HEPES/Na HEPES and 10 mM EDTA (pH 7.4), with mammalian protease inhibitor cocktail obtained from Sigma-Aldrich Corporation (St. Louis, Mo.), and brought into suspension in homogenization buffer using a cell scraper (GeneTools obtained from Philomath, Oreg.). The suspended cells are then homogenized and centrifuged (40,000×g for 10 min at 4° C.), and the resultant pellet is resuspended in homogenization buffer, and homogenized and centrifuged as before. The remaining membrane pellet is then twice washed and centrifuged in homogenization buffer, and the final membrane pellet resuspended in buffer comprising 20 mM HEPES/Na-HEPES and 0.1 mM EDTA (pH 7.4), and stored in the presence of bovine serum albumin (final concentration=1.0% (w/v)) at −135° C. The protein concentration in CHO-hGPR10 cell membranes is determined using a commercial Bradford assay (Sigma-Aldrich Corporation).

CHO-hGPR10 cell membranes thus prepared (20 μg protein) are incubated with 0.05 nM [¹²⁵I]PrRP (Amersham Biosciences UK Limited; catalog #IM341, Specific Activity=2000 Ci/mmol), test compound, and buffer comprising 20 mM Tris (pH 7.4), 2.0 mM EGTA, 5.0 mM C₄H₆MgO₄, 0.5 mM phenylmethylsulfonyl fluoride, 1.0 μg/ml pepstatin, 1.0 μl/ml protease inhibitor cocktail, and 0.1% (w/v) BSA. All of the reactions are performed in 96-well Basic Flashplateso (PerkinElmer Life Sciences), terminated by centrifugation (3,000 g×7 min) after a 90 min incubation at room temperature, and radioactive counts associated with membrane-bound [¹²⁵I]PrRP are counted in each well using a Microbeta TriLux microscintillation counter (PerkinElmer Life Sciences). Test compounds are classified as ‘hits’ if they displace ≧50% of specifically-bound [¹²⁵I]PrRP from CHO-hGPR10 cell membranes. This procedure can be used to screen 80 compounds at a time, 8 negative (vehicle; final concentration=0.5% (v/v) dimethyl sulfoxide) and 8 positive controls (PrRP; 1.0 nM final concentration) per 96-well Basic Flashplate®.

### In vitro Functional Assay for Detecting Compounds that Activate or Inhibit the Function of Cloned Wild-type or Truncated GPR10

Chinese hamster ovary cell lines are generated that stably express cloned wild-type (pcNDA3.1(+)-hGPR10:CHO-hGPR10 cells) and truncated forms of the human GPR10 (pcNDA3.1(+)-hGPR10-306:CHO-T-hGPR10), using the methods described in Examples 1-2. CHO-hDMO1 and CHO-T-hGPR10 cells are cultured until approximately 70% confluent and suspended in culture medium (150,000 cells/ml) containing 600 nCi/ml [³H]arachidonic acid (PerkinElmer Life Sciences; catalog #NET298, Specific Activity=189 Ci/mmol). 250 μl of this cell suspension is then added to all wells of two sterile 48-well plates and left to incubate for 17-24 hr at 37° C. On the day of the experiment, the culture medium is removed by aspiration and replaced with 250 μl of HBSS buffer comprising Ca²⁺ and Mg²⁺, 20 mM HEPES and 0.1% (w/v) fatty acid free BSA) and left to incubate for 5 min at 37° C. After a further 2 washes and incubations in HBSS buffer, 240 μl of HBSS buffer and 10 μl of either DMSO vehicle (final concentration=0.25% (v/v), PrRP, or PrRP mixed with test compound, are added to each well and plates are then left to incubate at room temperature on a 96-well plate shaker. In wells containing PrRP, the final concentration is always 1.0 nM. A 200 μl aliquot is then removed from each well and added to a Basic Flashplate®. The Basic Flashplate® is then sealed, shaken for 5 min at room temperature using a 96-well plate shaker, and then the amount of [³H]arachidonic acid present in each well is calculated using a Microbeta TriLux microscintillation counter. This procedure can be used to screen 80 compounds, 8 negative (vehicle; final concentration=0.5% (v/v) dimethyl sulfoxide) and 8 positive controls (PrRP; 1.0 nM final concentration) per 96-well Basic Flashplate®. Test compounds are classified as agonist hits if they increase basal [³H]arachidonic acid to >=190% of the mean effect of 1.0 nM PrRP, and as antagonist/inverse agonist hits if they inhibit basal [³H]arachidonic acid release to <=10% of the mean effect of 1.0 nM PrRP.

The following examples are provided for illustrative purposes only, and are in no way intended to limit the scope of the present invention.

### EXAMPLE 1

### Preparation of Recombinant Expression Vectors for Rat and Human GPR10 and Mutant GPR10

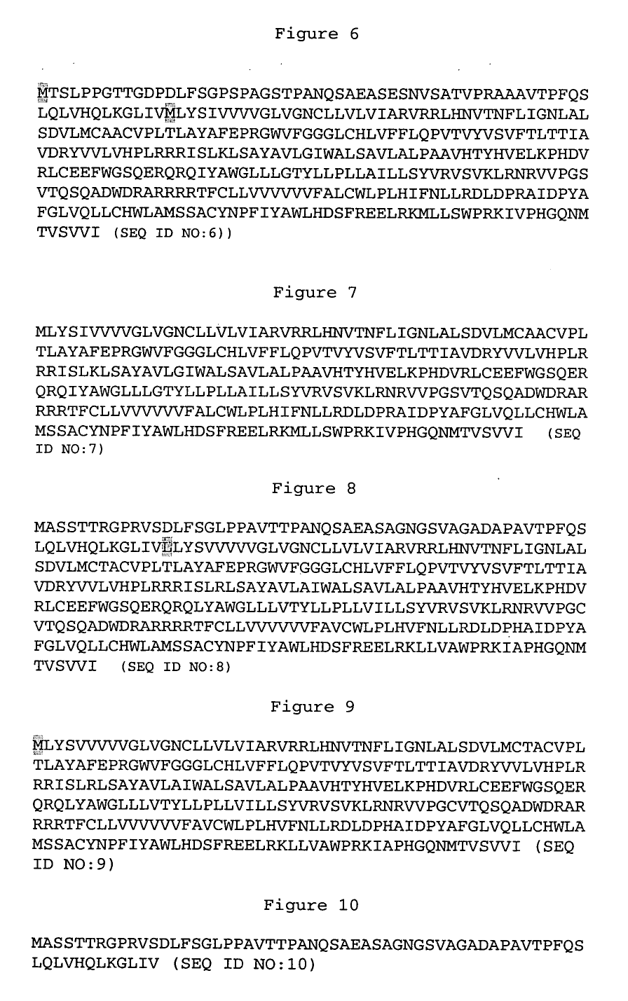

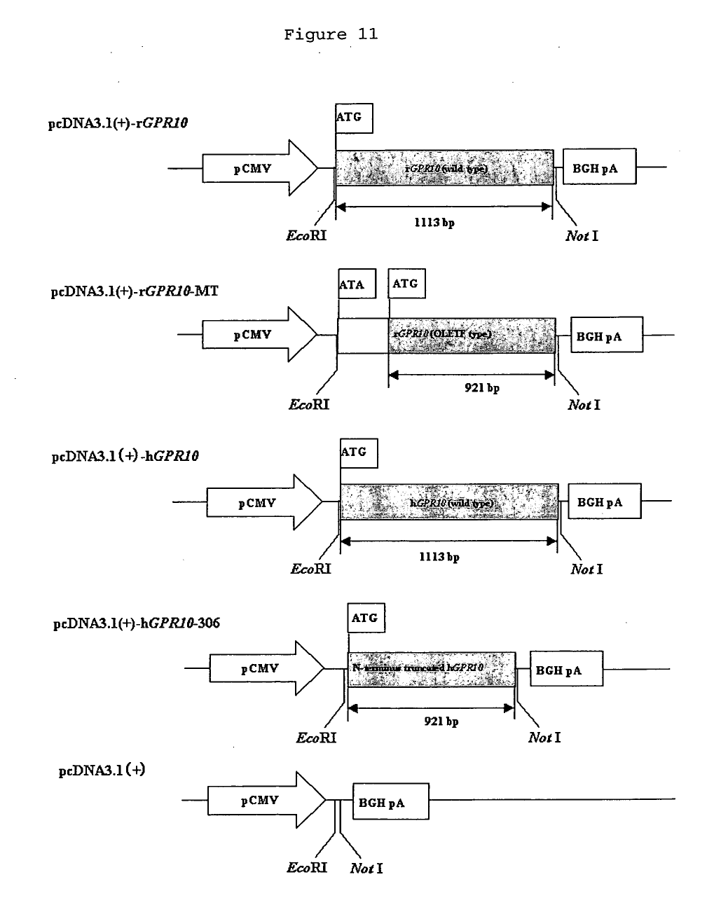

The nucleotide sequence of rat wild-type GPR10 and rat mutant GPR10 are shown in FIGS. 1-2, respectively. The amino acid sequences of rat wild-type GPR10 and rat mutant GPR10 are shown in FIGS. 6-7, respectively. The structure of the rat GPR10 expression vectors used herein are shown in FIG. 11.

For cloning of wild-type (WT) and mutant (MT) rat GPR10 ORF, primer sets were designed as follows: rGPR10WTF-EcoRI, 5′-ATGAATTCGTGGCCATGACCTCACTGCCC-3′ (SEQ ID NO:15) as the wild-type forward primer; rGPR10MTF-EcoRI, 5′-ATGAATTCGTGGCCATAACCTCACTGCCC-3′ (SEQ ID NO:16) as the mutant forward primer; rGPR10R-NotI, 5′-TAGCGGCCGCTCAGATGACCACACTGACGG-3′ (SEQ ID No. 17) as the common reverse primer.

Forward and reverse primers had EcoRI and NotI restriction enzyme sites at the 5′ ends, respectively (underlined). WT and MT GPR10 ORF were amplified from BN and OLETF rat genomic DNA as templates, respectively. These amplified fragments were digested with EcoRI and NotI restriction enzymes, and then inserted into the EcoRI and NotI site of pcDNA3.1(+) (Invitrogen). The WT and MT GPR10-expression vectors were named pcDNA3.1(+)-rGPR10 and pcDNA3.1(+)-rGPR10-MT, respectively.

The nucleotide sequence of human wild-type GPR10 and human mutant GPR10 are shown in FIGS. 4-5, respectively. The amino acid sequences of human wild-type GPR10 and human mutant GPR10 are shown in FIGS. 8-9, respectively. The structure of the human GPR10 expression vectors used herein are shown in FIG. 11.

For cloning of human wild-type GPR10 ORF, a DNA fragment was amplified from human genomic DNA (Promega; Catalog #G3041, Lot #88299) by PCR using primer sets as follows: hGPR10F-EcoRI, 5′-CGGAATTCGTGGCCATGGCCTCATCG-3′ (SEQ ID NO:18) as the forward primer; hGPR10R-NotI, 5′-CGAGCGGCCGCTCAGATGACCACGCTGAC-3′ (SEQ ID NO:19) as the reverse primer. The forward and reverse primers contained EcoRI and NotI restriction enzyme sites at the 5′ ends, respectively (underlined). The resulting amplified fragment was also inserted into pcDNA3.1(+). This expression vector was named pcDNA3.1(+)-hGPR10.

For construction of a human mutant GPR10 expression vector, the primer used was as follows: hGPR10/dell-F 5′-GTGAATTCATGCTCTACAGCGTCGTGGTG-3′ (SEQ ID NO:20). PCR was performed with hGPR10/dell-F and hGPRlOR-NotI primers. The amplified DNA fragment was inserted into the EcoRI and NotI restriction enzyme sites of pcDNA3.1(+) vector after digestion with EcoRI and NotI. This vector was named pcDNA3.1(+)-hGPR10-306.

### EXAMPLE 2

### Cloning of Rat and Human GPR10 and Mutant GPR10s in CHO Cells

For cloning of rat and human GPR10 and mutant GPR10 in CHO cells, a GPR10 stably expressing cell line was establishment as follows:

After linearization by digestion of 1.0 μg of each expression vector with ScaI, CHO-Kl cells (1.2×10⁵ cells/well in a 24-well plate) were transfected using 2.5 μl of LipofectAMINE2000 (Invitrogen) according to the supplemental protocol. One day after transfection, these transfectants were selected with selection medium comprising F-12 medium, 10% (v/v) FCS and 0.35 mg/ml of Geneticin, and single clones were isolated by the limited dilution method. The GPR10 expression levels for each cell were analyzed by Northern Blotting analysis.

### EXAMPLE 3

### Preparation of Congenic Rats Expressing Mutant GPR10

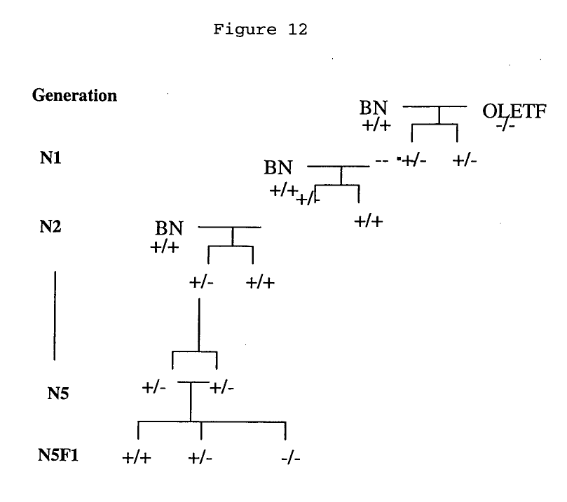

The generation of congenic rats expressing mutant GPR10 by crossing BN rats and OLETF rats is summarized in FIG. 12. Inbred BN rats were purchased from Charles River Japan Laboratories. The OLETF rats are described in U.S. Pat. No. 5,789,652, embryos of which were deposited under ATCC No. 72016.

The OLETF and BN rats were crossed, and the N1 rats were backcrossed to the BN rats to obtain the first generation of congenic rats (N2). For the N2 and subsequent generations, genomic DNA isolated from the tail tip was analyzed using microsatellite markers that distinguish the OLETF and BN alleles and confirmed OLETF-derived genome region from D1Rat169 to D1Rat459 remained and that other genome regions were replaced by the BN genome by 4 successive backcrosses (N5). At this N5 generation, the background genome of the rats had already been replaced by 97 to 99% of the BN genome. Heterozygous rats were selected and were subsequently intercrossed to establish the homozygous congenic rat. At each generation, genetic markers (D1Rat169, D1Rat305, D1Rat312, D1Rat910, D1Rat459; purchased from Research Genetics Inc) within the Dmol region were used to verify the integrity of the OLETF-susceptibility haplotype. Rats were genotyped by PCR amplification of microsatellite markers as described by Kanemoto et al, Mammalian Genome, 9:419-425 (1998). Furthermore, after the discovery of the GPR10 mutation in the OLETF rat, a PE Applied Biosystems 7700 Sequence Detector was used for the detection of GPR10 mutation (start codon: ATG→ATA) after PCR amplification using the TaqMan probe, as described above. Rats with a homozygous gene profile were subsequently used in a forced swimming test.

### EXAMPLE 4

### Forced Swimming Test

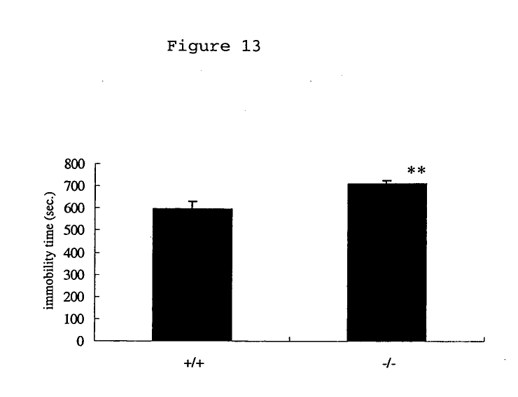

The forced swimming test (FST) was conducted according to the method of Porsolt et al, Eur. J. Pharmacol., 47:379 (1978), with minor modifications. Specifically, rats were individually forced to swim inside a vertical acrylic cylinder (height: 40 cm, diameter: 17 cm) containing 17.5 cm of water maintained at 23-25° C., and the total duration of immobility (immobility time) was measured for 15 min using an animal movement analyzing system (SCANET MV-10 AQ, TOYO SANGYO Co. Ltd.). After swimming, the rats were removed and allowed to dry under an electric lamp before being returned to their home cages. The next day, the rats were placed again in the cylinder for 5 min, and the immobility time was measured. The total immobility time was calculated from scanning data. The water in the tank was exchanged to avoid the deposit of animal pheromones and a decrease in the sensitivity of the sensor at every experiment. The result are shown in FIG. 13.

As shown in FIG. 13, congenic rats harboring the mutant GPR10 gene exhibited a significant increase in immobile time compared to that of the control +/+ rats in both the first FST (15 min) and the second FST (5 min). There are several factors that can be implied for this behavioral difference. First, it highlights a level of depression differing between the congenic rats and the control +/+ rats. As hypothesized by Porsolt et al, supra, this suggests the immobility condition reflects a state of lowered mood or hopeless. Thus, the congenic rats may suffer from depression more severely in comparison to control +/+ rats. Second, the sensitivity to swimming stress differs between the congenic rats and the control +/+ rats. As hypothesized by Hawkins et al, Nature, 274:512 (1978), behavioral immobility might be a consequence of adaptive response to a stressful situation, implying that the congenic rats are less fearful of swimming stress, compared with the control +/+ rats.

### EXAMPLE 5

### Elevated Plus-maze Test

In the elevated plus-maze test, a sense of security is provided by closed arms, whereas open arms offer exploratory value. Therefore, it is expected that anxious rats will spend less time in the open arms than those that are less fearful. The degree of anxiety is assessed by measuring the time spent in the open and closed arms, and the number of entries made into each arm. Benzodiazepines, barbiturates, and (sometimes) 5-HT1A receptor agonists are found to be anxiolytic in this test (Pellow et al, Neuroscience Methods, 14:149 (1985)). The test is also sensitive to anxiogenic drugs, which lends strong support for its predictive validity.

The elevated plus-maze test was used to assess anxiety in the control wild-type +/+ rats and in the congenic −/− rats to determine possible interfering emotional factors. The maze, which was elevated 60 cm above the floor, consisted of two opposing open arms [42×15 (width) cm] and two opposing closed arms [42×15×30 (wall height) cm] that extend from a central platform (15×15⁻ cm). Illumination intensities on the floor were 17-30 Lux at the open arms and 5-16 Lux at the closed arms. Rats were placed individually on the central platform, facing an open arm, and allowed to explore the apparatus for 5 min. Using a video-behavior analysis system, the behavior of the animals was recorded and scored-in automatically by designating the zones of the open arms, closed arms and central platform.

Open and closed arm entries, time spent in each arm, and total moving distances were scored for each rat. Rats were tested from 9:00 a.m. to 12:00 noon. The test results are summarized in Table 1 below.

TABLE 1

| | +/+ rat | −/− rats |
|---|---|---|
| N= | 10 | 8 |
| Open arm Entries (counts) | 2.9 ± 0.82 | 3.9 ± 0.85 |
| Stay (seconds) | 17.9 ± 4.82 | 47.8 ± 11.9* |
| Closed arm Entries (counts) | 3.9 ± 0.82 | 4.9 ± 0.85 |
| Stay (seconds) | 282.1 ± 4.82 | 252.3 ± 11.9* |
| Total moving distance (cm) | 72.2 ± 9.61 | 111.0 ± 9.40* |

For comparison between the congenic −/− rat group and the control +/+ rat group, the F-test was employed to detect differences in the variance.

If the variance was homogeneous, the Student's t-test was used, and if not, the Aspin-Welch t-test was used. * P<0.05 by the Aspin-Welch t-test in the open arm stay time and Closed arm stay time. * P<0.05 by the Student's t-test in terms of the total moving distance.

As shown in Table 1 above, the congenic −/− rats showed a prolonged time spent in the open arms, and a shorter time spent in the closed arms compared with control +/+ rats. Also, the total moving distance was longer in the congenic −/− rats than in the control +/+ rats. These results suggest that the congenic −/− rats are less fearful than the control +/+ rats.

### EXAMPLE 6

### Effect of PrRP and Compound X on [¹²⁵I]PrRP Binding to CHO Cells Stably Expressing Cloned Human Wild-type and Mutant GPR10

Chinese hamster ovary cell lines were generated that stably expressed wild-type (pcDNA3.1(+)-hGPR10:CHO-hGPR10 cells) or a mutant form (pcDNA3.1(+)-hGPR10-306: CHO-T-hGPR10; having a truncation of the first 64 amino acids of the NH₂-terminus) of the human GPR10 receptor, as described in Examples 1-2. Membranes were prepared from CHO-hGPR10 and CHO-T-hGPR10 cells using the following procedure.

Each cell line was cultured until approximately 70% confluent, washed three times with ice-cold homogenization buffer comprising 20 mM HEPES/Na HEPES and 10 mM EDTA (pH 7.4), with mammalian protease inhibitor cocktail—Sigma-Aldrich Corporation (St. Louis, Mo.), and brought into suspension in homogenization buffer using a cell scraper (GeneTools—Philomath, Oreg.). Suspended cells were then homogenized and centrifuged (40,000×g for 10 minutes at 4° C.), and the resultant pellet was resuspended in homogenization buffer, and homogenized and centrifuged as before. The remaining membrane pellet was then twice washed and centrifuged in homogenization buffer, and the final membrane pellet was resuspended in buffer comprising 20 mM HEPES/Na-HEPES and 0.1 mM EDTA (pH 7.4), and stored in the presence of bovine serum albumin (final concentration=1.0% (w/v)) at −135° C. The protein concentration in CHO-hGPR10 and CHO-T-hGPR10 membranes was determined using a commercial Bradford assay (Sigma-Aldrich Corporation).

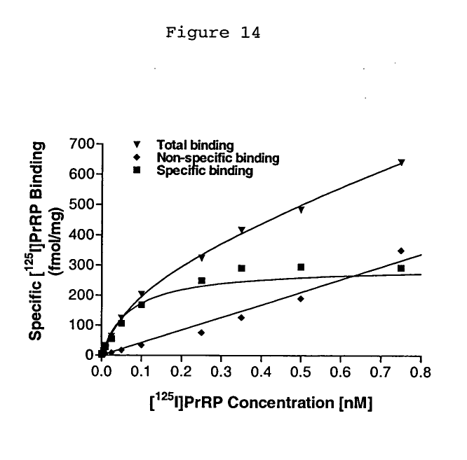

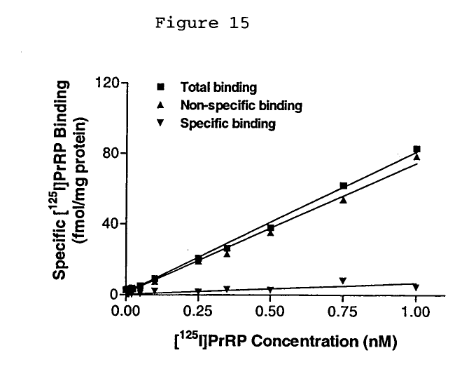

Then, saturation binding assays were performed to estimate a dissociation constant (K_{D}) for [¹²⁵I]PrRP binding to CHO-hGPR10 and CHO-T-hGPR10 cell membranes, as well as the density of receptor expression (B_{MAX}) in each membrane preparation. Both saturation binding assays were conducted using the following procedure.

[¹²⁵I]PrRP (Amersham Biosciences UK Limited; catalog #IM341, Specific Activity=2000 Ci/mmol) was evaluated in quadruplicate at 9 different concentrations (0.005, 0.01, 0.025, 0.05, 0.10, 0.25, 0.35, 0.50 and 0.75 nM) in buffer comprising 20 mM Tris (pH 7.4), 2.0 mM EGTA, 5.0 mM C₄H₆MgO₄, 0.5 mM phenylmethylsulfonyl fluoride, 1.0 μg/ml pepstatin, 1.0 μl/ml protease inhibitor cocktail, and 0.1% (w/v) BSA (all of the buffer ingredients were purchased from Sigma-Aldrich Corporation), mixed with 20 μg of CHO-hGPR10 or CHO-T-hGPR10 membrane protein. Non-specific binding was determined in the presence of 1.0 μM PrRP. All of the reactions were performed in 96-well Basic Flashplates®, terminated by centrifugation (3,000 g×7 min) following a 90 min incubation at room temperature, and radioactive counts associated with membrane-bound [¹²⁵I]PrRP were counted in each well using a Microbeta TriLux microscintillation counter (PerkinElmer life Sciences (Boston, Mass.)). Estimates of K_{D} and B_{MAX} were calculated by computerized non-linear regression analysis of complete saturation binding data using GraphPad Prism version 3.00 for Windows (GraphPad Software, San Diego, Calif.). The results are shown in FIGS. 14-15.

As shown in FIG. 14, the saturation analysis of this data conformed to a 2-site competition binding model (goodness of fit, R²=0.87), whereby [¹²⁵I]PrRP binds to two high affinity sites (K_{D1}=0.070; K_{D2}=0.072 nM) expressed at a similar density (B_{MAX1}=147.5 nM; B_{MAX2}=147.4 nM) on CHO-hGPR10 cell membranes. [¹²⁵I]PrRP binding was saturable and at [¹²⁵I]PrRP concentrations that approached the estimated KD values, more than 80% of total [¹²⁵I]PrRP binding was specific. In comparison, as shown in FIG. 15, [¹²⁵I]PrRP bound in a non-specific and non-saturable manner to CHO-T-hGPR10 membranes, demonstrating that [¹²⁵I]PrRP did not bind to this mutant form of GPR10. Taken together, these results provide direct evidence that the [¹²⁵I]PrRP binding site resides within the first 64 amino acids of the NH₂-terminus of the human GPR10 receptor, and this binding site consists of two binding domains that contact [¹²⁵I]PrRP each at a subnanomolar binding affinity.

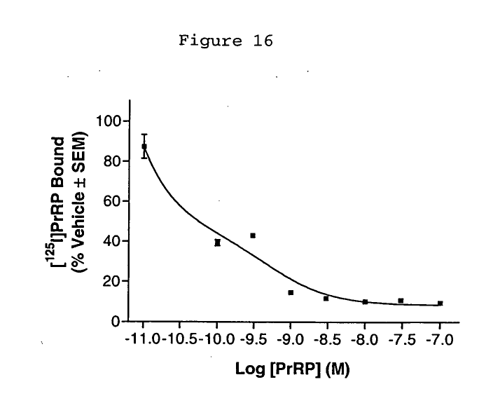

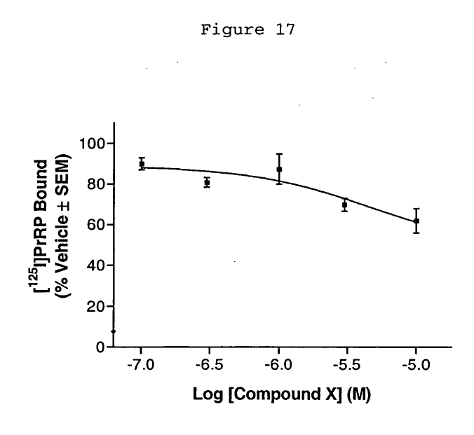

Next, PrRP and compound X (molecular weight=318.38) were studied in quadruplicate at nanomolar to micromolar concentrations (PrRP at 0.01, 0.1, 0.5, 1, 5, 10, 50 and 100 nM; Compound X at 100, 300, 1000, 3000 and 10,000 nM) to determine their respective displacement of [¹²⁵I]PrRP (Amersham Biosciences UK Limited; catalog #IM341, Specific Activity=2000 Ci/mmol) binding to CHO-hGPR10 cell membranes. Membranes (20 μg protein) were incubated with 0.05 nM [¹²⁵I]PrRP, Compound X or vehicle, and buffer comprising 20 mM Tris (pH 7.4), 2.0 mM EGTA, 5.0 mM C₄H₆MgO₄, 0.5 mM phenylmethylsulfonyl fluoride, 1.0 μg/ml pepstatin, 1.0 μl/ml protease inhibitor cocktail, and 0.1% (w/v) BSA. All of the reactions were performed in 96-well Basic Flashplates®, terminated by centrifugation (3,000 g×7 min) after a 90 min incubation at room temperature, and radioactive counts associated with membrane-bound [¹²⁵I]PrRP were counted in each well using a Microbeta TriLux microscintillation counter. Non-linear regression analysis of the competition binding data was used to calculate inhibition binding constants (IC₅₀ and Kᵢ), which is the concentration of Compound X that occupies half of the CHO-hGPR10 receptors specifically bound by 0.05 nM [¹²⁵I]PrRP. The binding affinity of CHO-hGPR10 receptors for Compound X (Ki) was calculated by the equation, Kᵢ=(IC₅₀)/(1+([[¹²⁵I]PrRP]/K_{D}), where the K_{D} for [¹²⁵I]PrRP at CHO-hGPR10=0.07 nM, was calculated in the saturation binding assay described above. Binding affinity estimates for PrRP and Compound X at the CHO-hGPR10 receptor were calculated using GraphPad Prism version 3.00 for Windows. The results are shown in FIG. 16-17.

As shown in FIG. 16, increasing concentrations of PrRP competitively displaced [¹²⁵I]PrRP binding to CHO-hGPR10 cell membranes. These displacement data closely conformed (Goodness of fit (R²)=0.97) to a 2-site competition model that estimated PrRP displaced [¹²⁵I]PrRP binding from two subnanomolar affinity sites (IC_{50 (1)}=0.0007, K_{I (1)}=0. 0004 nM, IC_{50 (2)}=0.50 nM, K_{i (2)}=0.29 nM) on CHO-hGPR10 membranes. These results are consistent with the 2 site binding of [¹²⁵I]PrRP to CHO-hGPR10 estimated by saturation binding. In contrast, however, as shown in FIG. 17, Compound X displaced approximately 40% of specifically bound [¹²⁵I]PrRP from a single site on CHO-hGPR10 membranes, Goodness of fit (R²)=0.86. IC₅₀=4.63 μM, Kᵢ=2.70 μM. This difference between displacement of [¹²⁵I]PrRP binding by PrRP and Compound X may be explained by (1) Compound x competitively displacing [¹²⁵I]PrRP from one of the 2 binding sites present on the NH₂-terminus of CHO-hGPR10, or (2) Compound X may bind to a distinct intracellular sequence of CHO-hGPR10, and such binding may effect a conformational change in CHO-hGPR10 that results in non-competitive displacement of [¹²⁵I]PrRP binding from the extracellular NH₂-terminus of CHO-hGPR10. Based on the relatively large difference in molecular weight, size, and overall surface charge between PrRP, a peptide, and Compound X, a small molecule, it is believed that Compound X competitively displaces [¹²⁵I]PrRP by occupying one of the two [¹²⁵I]PrRP binding sites on the NH₂-terminus of CHO-hGPR10. Small molecule displacement of peptide binding to other GPR's is most likely, if not always, a result of non-competitive peptide displacement following small molecule occupation of an intracellular domain located remotely to the extracellular NH₂-terminus peptide binding (J. Biol. Chem., 274:8694 (1999)). A similar allosteric, non-competitive displacement is believed to enable Compound X to non-competitively displace [¹²⁵I]PrRP.

### EXAMPLE 7

### Effect of PrRP and Compound X on [³H]arachidonic Acid Release from CHO Cells Stably Expressing Cloned Human Wild-type GPR10

CHO cells stably expressing cloned human wild-type GPR10 (CHO-hGPR10 cells) were generated as described in Examples 2-3. CHO-hGPR10 were cultured in F-12 HAM medium supplemented with 2.0 mM L-glutamine, 10% (v/v) dialyzed fetal bovine serum, 1.0% hypoxanthine-thymidine supplement, and 1.0% G-418 selection agent. All of the cells were maintained at 37° C. in an atmosphere of 5% CO₂ and passaged when ˜80% confluent using a non-enzymatic cell dissociation buffer (Sigma-Aldrich Corporation). CHO-hGPR10 cells were harvested when ˜70% confluent for use in [³H]arachidonic acid release assays, which were performed as follows:

CHO-hGPR10 cells were plated at a density of 150,000 cells/well in 12 well tissue culture plates, rinsed 24 hr later with calcium and magnesium free Hank's Balanced Salt Solution (Gibco-BRL), and left to incubate for 4 hr in F-12 culture medium (1.0 ml/well) containing 100 nCi/ml of [³H]arachidonic acid. The cells were then rinsed and incubated for 5 min in 1.0 ml/well of Hank's Balanced Salt Solution (Invitrogen Corp. CA) containing 20 mM HEPES buffer (Sigma-Aldrich Corp.) and 0.1% (w/v) fatty acid-free bovine serum albumin (Sigma-Aldrich Corp.). After repeating this rinse and incubation procedure 3 times, 1.0 ml of vehicle, PrRP or Compound X was added to each well and plates were left to incubate for 30 min at 37° C. 400 μl of the extracellular liquid present in each well was mixed with 2.6 ml of liquid scintillation cocktail and counted by liquid scintillation counting (1272 Clinigamma, LKB/Wallach) to calculate the total [³H] radioactivity present in each well. All of the reactions were performed in triplicate, at concentrations in the 0.01 to 10,000 nM range, to determine the effects of PrRP and Compound X on basal [³H]arachidonic acid release from CHO-hGPR10 cells.

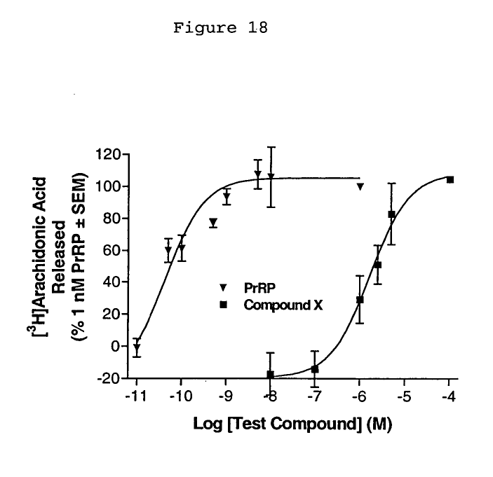

The mean radioactive counts (±SEM, N=3) detected in each reaction triplicate were calculated, and binding isotherms were analyzed by non-linear regression, using GraphPad Prism version 3.00 for Windows, to estimate the potency (EC₅₀ with 95% confidence interval) and relative intrinsic activity (Eₘₐₓ, as a % of 1 nM PrRP±SEM) for PrRP and Compound X. The results are shown in FIG. 18.

As shown in FIG. 18, PrRP and Compound X both stimulated concentration-dependent increases in [³H]arachidonic acid release from CHO-hGPR10 cells, and while PrRP (EC₅₀=0.043 nM, 0.01 to 0.19 nM; Eₘₐₓ=105.4±6.1%) was more potent than Compound X (EC₅₀=1.68 μM, 0.95 to 2.97 μM; Eₘₐₓ=108.4±5.4%), both compounds displayed similar relative intrinsic activities. The potency of both PrRP and Compound X in this study are consistent with the respective subnanomolar and low micromolar binding affinities demonstrated for PrRP and Compound X in the [¹²⁵I]PrRP competition binding assay using CHO-hGPR10 cell membranes. This present study demonstrates the utility of an in vitro [³H]arachidonic acid release assay to measure agonistic drug activity on cloned human wild-type GPR10 heterologously expressed in CHO cells.

### EXAMPLE 8

### Effects of PrRP and Compound X on [³H]arachidonic Acid Release from CHO Cells Stably Expressing Cloned Human Mutant GPR10

CHO cells were generated to stably express a cloned truncated form of the human GPR10, as described in Examples 2-3. These CHO-T-hGPR10 cells, which express a truncation of the first 64 amino acids present in the wild-type cloned human GPR10, were cultured in F-12 HAM medium, supplemented with 2.0 mM L-glutamine, 10% (v/v) dialyzed fetal bovine serum, 1.0% hypoxanthine-thymidine supplement and 1.0% G-418 selection agent. All of the cells were maintained at 37° C. in an atmosphere of 5% CO₂ and passaged when ˜80% confluent using a non-enzymatic cell dissociation buffer (Sigma-Aldrich Corporation). CHO-T-hGPR10 cells were harvested when ˜70% confluent for use in [³H]arachidonic acid release assays carried out as described below.

The effects of PrRP and Compound X on basal [³H]arachidonic acid release from CHO-T-hGPR10 cells was performed using exactly the same assay procedure described in Example 7. PrRP and Compound X were each tested in triplicate at concentrations in the 0.01 to 10,000 nM range.

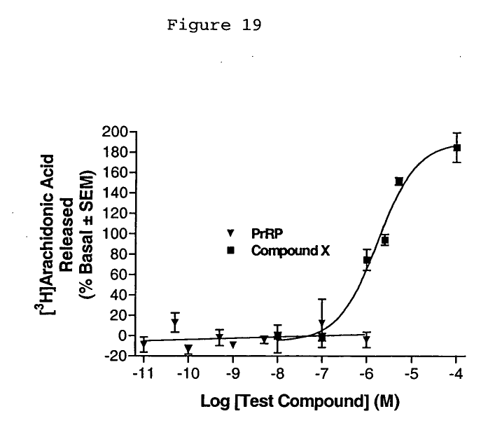

The mean radioactive counts (±SEM, N=3) detected in each reaction triplicate were calculated, and binding isotherms were analyzed by non-linear regression, using GraphPad Prism version 3.00 for Windows, to estimate the potency (EC₅₀ with 95% confidence interval) and relative intrinsic activity (Eₘₐₓ as % basal release±SEM) of PrRP and Compound X. The results are shown in FIG. 19.

As shown in FIG. 19, PrRP was inactive in this assay of CHO-T-hGPR10 cell function, although Compound X concentration-dependently increased basal [³H]arachidonic acid release in CHO-T-hGPR10 cells (EC₅₀=1.73 μM, 0.70 to 4.25 μM; Eₘₐₓ=189.9±13.3%). The contrasting activity and inactivity of PrRP in CHO-hGPR10 and CHO-T-hGPR10 cells, respectively, demonstrate that PrRP activates GPR10 at a location within the first 64 amino acids of the NH₂-terminus of the cloned human wild-type GPR10. This conclusion is consistent with the [¹²⁵I]PrRP saturation and competition binding studies, which revealed that PrRP binds specifically to CHO-hGPR10 (FIG. 14), but not CHO-T-hGPR10 cells (FIG. 15) as described in Example 6. In contrast, Compound X stimulated [³H]arachidonic acid release from CHO-T-hGPR10 and CHO-T-hGPR10 cells, suggesting Compound X activates GPR10 at a location outside of the first 64 amino acids of the NH₂-terminus of the cloned human wild-type GPR10. Together these data serve as in vitro evidence that the orthosteric binding site of the cloned human GPR10 is located within the first 64 amino acids of the NH₂-terminus, as the endogenous peptide agonist PrRP was active only active in cells containing the wild-type, but not the truncated NH₂-terminus form of GPR10. The existence of an allosteric binding site located elsewhere within the same receptor is also suggested by the activity of Compound X in cells expressing the wild-type and truncated forms of GPR10. More potent analogs of Compound X may provide therapeutic benefit to diseased states associated with deficient GPR10-mediated signaling that may arise, for example, from genetic abnormalities in GPR10 expression or signal transduction.

### EXAMPLE 9

### Synergistic Effect of Compound X on PrRP-stimulated [³H]arachidonic Acid Release from CHO Cells Stably Expressing Cloned Human Wild-type GPR10

Example 8 suggests that PrRP and Compound X activate CHO-hGPR10 cells by respective activation at an orthosteric and allosteric site located at different positions within the cloned human GPR10 receptor. The present study was designed to investigate whether PrRP-mediated [³H]arachidonic acid release from CHO-hGPR10 cells is affected by increasing concentrations of Compound X. Compound X was tested in triplicate at concentrations in the 0.01 to 10,000 nM range in the presence and absence of 1.0 nM PrRP.

CHO cells were generated to stably express a cloned human wild-type GPR10, as described in Examples 2-3. These CHO-hGPR10 cells were cultured in F-12 HAM medium, supplemented with 2.0 mM L-glutamine, 10% (v/v) dialyzed fetal bovine serum, 1.0% hypoxanthine-thymidine supplement and 1.0% G-418 selection agent. All of the cells were maintained at 37° C. in an atmosphere of 5% CO₂ and passaged when ˜80% confluent using a non-enzymatic cell dissociation buffer (Sigma-Aldrich Corporation). CHO-hGPR10 cells were harvested when ˜70% confluent for use in [³H]arachidonic acid release assays, which were carried out as described above.

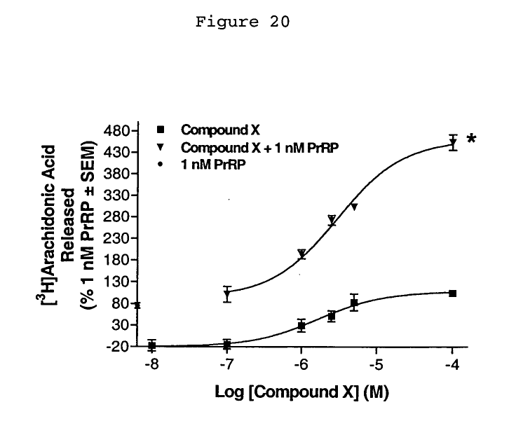

The mean radioactive counts (±SEM, N=3) detected in each reaction triplicate were calculated, and binding isotherms were analyzed by non-linear regression, using GraphPad Prism version 3.00 for Windows, to estimate the potency (EC₅₀ with 95% confidence interval) and relative intrinsic activity (Eₘₐₓ, as % 1.0 nM PrRP±SEM) of Compound X tested in the presence and absence of 1.0 nM PrRP. An unpaired t-test was used to detect statistically significant (P<0.05) differences between the effects of Compound X and Compound X+1.0 nM PrRP upon basal [³H]arachidonic acid release in CHO-hGPR10 cells. The results are shown in FIG. 20.

As shown in FIG. 20, when Compound X was combined with 1.0 nM PrRP, the resulting response was twice as potent as Compound X tested alone. Moreover, Compound X exhibited a significantly (P<0.0001) 4-fold higher relative intrinsic activity than that produced by either 1.0 nM PrRP or Compound X+1.0 nM PrRP (Compound X, EC₅₀=1.68 μM, 0.95 to 2.96 μM; Eₘₐₓ=108.4±5.4%. Compound X+1.0 nM PrRP, EC₅₀=3.0 μM, 1.17 to 7.74 μM; Eₘₐₓ=459.0±18.8%, 1.0 μM PrRP, Eₘₐₓ=75.3±6.1%). Moreover, this interaction was clearly synergistic, as the sum of the relative intrinsic activities for 1.0 nM PrRP and Compound X were 2.5-fold lower than the relative intrinsic activity of Compound X tested in the presence of 1.0 nM PrRP. This synergistic interaction between PrRP and Compound X is believed to result from the combined respective activities of these compounds at an orthosteric and allosteric site located at distinct regions within the cloned human GPR10, as proposed as a result of the findings in Example 8.

### EXAMPLE 10

### Effects of Compound X on PrRP-stimulated [³H]arachidonic Acid Release from CHO Cells Stably Expressing Cloned Human Mutant GPR10

Examples 8 and 10 suggest that PrRP and Compound X activate CHO-hGPR10 cells by respective activation at an orthosteric and allosteric site located at different positions within the cloned human GPR10. The present study was designed to determine the importance of the proposed orthosteric NH₂-terminus binding site in mediating the synergistic interaction between PrRP and Compound X, as described in Example 9. For this study, Compound X was tested in triplicate, at concentrations in the 0.01 to 10,000 nM range, for its effects on basal [³H]arachidonic acid release from CHO-T-hGPR10 cells.

CHO cells were generated to stably express a cloned truncated form of the human GPR10, as described in Examples 2-3. These CHO-T-hGPR10 cells, expressing a truncation of the first 64 amino acids present in the wild-type cloned human GPR10, were cultured in F-12 HAM medium, supplemented with 2.0 mM L-glutamine, 10% (v/v) dialyzed fetal bovine serum, 1.0% hypoxanthine-thymidine supplement and 1.0% G-418 selection agent. All of the cells were maintained at 37° C. in an atmosphere of 5% CO₂ and passaged when ˜80% confluent using a non-enzymatic cell dissociation buffer (Sigma-Aldrich Corporation). CHO-T-hGPR10 cells were harvested when ˜70% confluent for use in [³H]arachidonic acid release assays, as described above.

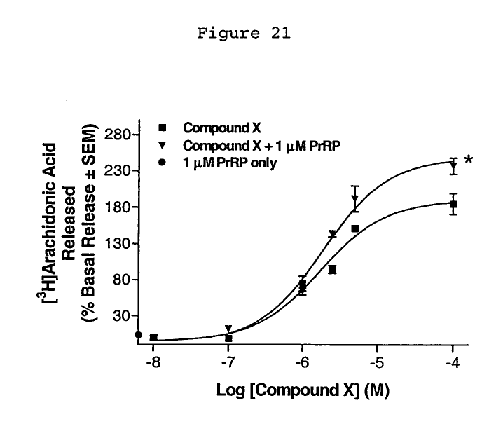

The mean radioactive counts (±SEM, N=3) detected in each reaction triplicate were calculated, and binding isotherms were analyzed by non-linear regression, using GraphPad Prism version 3.00 for Windows, to estimate the potency (EC₅₀ with 95% confidence interval), and relative intrinsic activity (Eₘₐₓ, as % basal release±SEM) of Compound X tested in the presence and absence of 1.0 nM PrRP. An unpaired t-test was used to detect statistically significant (P<0.05) differences between the effects of Compound X and Compound X+1.0 nM PrRP upon basal [³H]arachidonic acid release in CHO-T-hGPR10 cells. The results are shown in FIG. 21.

As shown in FIG. 21, Compound X concentration-dependently increased basal [³H]arachidonic acid release in CHO-T-hGPR10 cells (EC₅₀=1.73 μM, 0.70 to 4.25 μM; Eₘₐₓ=189.9±13.3%), and the relative intrinsic activity of this response was significantly elevated by 30% (P<0.0001) when Compound X was tested in the presence of 1.0 nM PrRP (EC₅₀=1.88 μM, 0.47 to 7.46 μM; Eₘₐₓ=248.1±17.7%). This enhancement of Compound X activity by PrRP represents a potentiative interaction between PrRP and Compound X, as 1.0 nM PrRP alone did not affect basal [³H]arachidonic acid release in CHO-T-hGPR10 cells (Eₘₐₓ=3.6±4.4%). These results demonstrate that PrRP has the capacity to enhance the activity of Compound X, although this effect is greatly diminished in CHO-T-hGPR10 cells lacking the proposed orthosteric binding site responsible for both PrRP-induced activation of cloned human GPR10 function and for the 4-fold synergistic effect of PrRP on Compound X-induced activation of cloned human GPCR activity.

While the invention has been described in detail and with reference to specific embodiments thereof, it will be apparent to one skilled in the art that various changes and modifications can be made therein without departing from the spirit and scope thereof.

What is claimed is:

1. A congenic rat comprising a mutant GPR10 gene, wherein said congenic rat is obtained by crossing a Otsuka Long-Evans Tokushima Fatty (OTELF) rat (ATCC No. 72016) with a wild-type rat, and wherein said congenic rat exhibits a prolonged immobilization time when assayed in a forced swim test compared to said wild-type rat and anti-anxiety behavior in a elevated plus-maze test compared to said wild-type rat.

2. The congenic rat of claim 1, wherein said mutant GPR10 gene consists essentially of the DNA sequence of SEQ ID NO:2.

3. The congenic rat of claim 1, wherein said wild-type rat comprises a GPR10 gene having the DNA sequence of SEQ ID NO:1.

4. The congenic rat of claim 1, wherein said OTELF rat comprises said mutant GPR10 gene.

5. A tissue or cell obtained from the congenic rat of claim 1, wherein said tissue and cell express said mutant GPR10 gene.

6. A culture of the cell of claim 5.

7. An isolated DNA molecule encoding a mutant GPR10 protein consisting essentially of the amino acid sequence of SEQ ID NO:7 or SEQ ID NO:9.

8. The isolated DNA molecule of claim 7, wherein said DNA molecule consists essentially of the DNA sequence of SEQ ID NO:2 or SEQ ID Nb:5.

9. An isolated fragment of human GPR10 consisting essentially of the amino acid sequence of SEQ ID NO:10.

10. A method for screening for a compound that inhibits GPR10 protein activity comprising:
(a) contacting a test compound with:
(i) the congenic rat of claim 1, and
(ii) the wild-type rat of claim 1; and
(b) assaying for GPR10 activity in the resulting rats (i) and (ii),
wherein when said activity is found to differ in said rats (i) and (ii), said test compound is identified as a compound that inhibits GPR10 protein activity.

11. The method of claim 10, wherein said GPR10 activity is depression in a forced swimming test or fear and anxiety in an elevated plus-maze test.

12. A method for screening for a compound that inhibits GPR10 protein activity comprising:
(a) contacting a test compound with:
(i) the tissue or cell of claim 5 or the isolated protein of claim 9, and with
(ii) the tissue or cell obtained from a wild-type rat which expresses wild-type GPR10 protein or wild-type GPR10 protein, respectively; and
(b) assaying for GPR10 activity in the resulting tissues or cells or isolated proteins (i) and (ii),
wherein when said activity is found to differ in said tissues or cells or isolated proteins (i) and (ii), respectively said test compound is identified as a compound that inhibits GPR10 protein activity.

13. The method of claim 12, wherein said GPR10 protein activity is binding of PrRP as measured in a competition assay, or release of arachidonic acid metabolite.

14. A method for screening for a compound useful in treating depression or anxiety comprising:
(a) administering the test compound identified as described in claim 10 or 12 as a compound that inhibits GPR10 activity, to a mammal suffering from depression or anxiety; and
(b) assaying for amelioration of said depression or anxiety in the resulting mammal so as to identify a compound useful in treating depression or anxiety.


SEQUENCE LISTING (20 sequences)

SEQ ID NO: 1 - DNA, 1113 bp, Rattus sp.
atgacctcactgccccctggaaccactggggaccccgatttgttttctgggccgtcgccagccggctccactccagccaaccagagtgcagaggcttcagagagcaatgtgtctgcgacggttcccagagctgcagcagtcacgccgttccagagcctgcaactagtgcaccagctgaagggactgatcgtgatgctgtacagcatcgtggtggtcgtgggtctggtgggcaactgccttcttgtgctggtgatcgcgcgcgtgcgccggctgcacaacgtgaccaacttcctcatcggcaacctggccttgtccgatgtgctcatgtgtgccgcctgtgtgcctctcacgctggcctacgcctttgaacctcgtggctgggtgttcggtggaggcctgtgccaccttgttttcttcctgcagccggtcaccgtctacgtatcggtgttcacactcaccacaatcgctgtggaccgctatgtggttctggtgcacccgctacgtcggcgcatttcactgaagctcagcgcctacgctgtgctgggcatctgggctctatctgcagtgctggcgctgccggccgcggtgcacacctaccatgtagagctcaagccccacgacgtgcgcctctgcgaggagttctggggttcgcaggagcgccagcgacagatctatgcctgggggctgctgctgggcacctatttgctccccctgctggccattctcctgtcttacgtccgggtgtcggtgaagttgcggaaccgcgtggtgcctggcagcgtgacccagagccaggctgactgggaccgagcgcgtcgccgtcgcactttctgcctgctggtggtggtggtggtcgtgttcgcggtctgctggctgcctctgcacattttcaacctgctgcgggacctggacccgcgtgccatcgacccctacgccttcgggctggtgcagctcctctgccactggcttgccatgagctccgcctgctacaaccccttcatctatgcgtggctgcacgacagcttccgagaggagctacgcaagatgcttctgtcttggccccgcaagatcgtgcctcatggccagaatatgaccgtcagtgtggtcatctga

SEQ ID NO: 2 - DNA, 1113 bp, Rattus sp.
ataacctcactgccccctggaaccactggggaccccgatttgttttctgggccgtcgccagccggctccactccagccaaccagagtgcagaggcttcagagagcaatgtgtctgcgacggttcccagagctgcagcagtcacgccgttccagagcctgcaactagtgcaccagctgaagggactgatcgtgatgctgtacagcatcgtggtggtcgtgggtctggtgggcaactgccttcttgtgctggtgatcgcgcgcgtgcgccggctgcacaacgtgaccaacttcctcatcggcaacctggccttgtccgatgtgctcatgtgtgccgcctgtgtgcctctcacgctggcctacgcctttgaacctcgtggctgggtgttcggtggaggcctgtgccaccttgttttcttcctgcagccggtcaccgtctacgtatcggtgttcacactcaccacaatcgctgtggaccgctatgtggttctggtgcacccgctacgtcggcgcatttcactgaagctcagcgcctacgctgtgctgggcatctgggctctatctgcagtgctggcgctgccggccgcggtgcacacctaccatgtagagctcaagccccacgacgtgcgcctctgcgaggagttctggggttcgcaggagcgccagcgacagatctatgcctgggggctgctgctgggcacctatttgctccccctgctggccattctcctgtcttacgtccgggtgtcggtgaagttgcggaaccgcgtggtgcctggcagcgtgacccagagccaggctgactgggaccgagcgcgtcgccgtcgcactttctgcctgctggtggtggtggtggtcgtgttcgcggtctgctggctgcctctgcacattttcaacctgctgcgggacctggacccgcgtgccatcgacccctacgccttcgggctggtgcagctcctctgccactggcttgccatgagctccgcctgctacaaccccttcatctatgcgtggctgcacgacagcttccgagaggagctacgcaagatgcttctgtcttggccccgcaagatcgtgcctcatggccagaatatgaccgtcagtgtggtcatctga

SEQ ID NO: 3 - DNA, 192 bp, Homo sapiens
atggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtg

SEQ ID NO: 4 - DNA, 1110 bp, Homo sapiens
atggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatc

SEQ ID NO: 5 - DNA, 921 bp, Homo sapiens
atgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatctga

SEQ ID NO: 6 - protein, 370 aa, Rattus sp.
MTSLPPGTTGDPDLFSGPSPAGSTPANQSAEASESNVSATVPRAAAVTPFQSLQLVHQLKGLIVMLYSIVVVVGLVGNCLLVLVIARVRRLHNVTNFLIGNLALSDVLMCAACVPLTLAYAFEPRGWVFGGGLCHLVFFLQPVTVYVSVFTLTTIAVDRYVVLVHPLRRRISLKLSAYAVLGIWALSAVLALPAAVHTYHVELKPHDVRLCEEFWGSQERQRQIYAWGLLLGTYLLPLLAILLSYVRVSVKLRNRVVPGSVTQSQADWDRARRRRTFCLLVVVVVVFALCWLPLHIFNLLRDLDPRAIDPYAFGLVQLLCHWLAMSSACYNPFIYAWLHDSFREELRKMLLSWPRKIVPHGQNMTVSVVI

SEQ ID NO: 7 - protein, 306 aa, Rattus sp.
MLYSIVVVVGLVGNCLLVLVIARVRRLHNVTNFLIGNLALSDVLMCAACVPLTLAYAFEPRGWVFGGGLCHLVFFLQPVTVYVSVFTLTTIAVDRYVVLVHPLRRRISLKLSAYAVLGIWALSAVLALPAAVHTYHVELKPHDVRLCEEFWGSQERQRQIYAWGLLLGTYLLPLLAILLSYVRVSVKLRNRVVPGSVTQSQADWDRARRRRTFCLLVVVVVVFALCWLPLHIFNLLRDLDPRAIDPYAFGLVQLLCHWLAMSSACYNPFIYAWLHDSFREELRKMLLSWPRKIVPHGQNMTVSVVI

SEQ ID NO: 8 - protein, 370 aa, Homo sapiens
MASSTTRGPRVSDLFSGLPPAVTTPANQSAEASAGNGSVAGADAPAVTPFQSLQLVHQLKGLIVLLYSVVVVVGLVGNCLLVLVIARVRRLHNVTNFLIGNLALSDVLMCTACVPLTLAYAFEPRGWVFGGGLCHLVFFLQPVTVYVSVFTLTTIAVDRYVVLVHPLRRRISLRLSAYAVLAIWALSAVLALPAAVHTYHVELKPHDVRLCEEFWGSQERQRQLYAWGLLLVTYLLPLLVILLSYVRVSVKLRNRVVPGCVTQSQADWDRARRRRTFCLLVVVVVVFAVCWLPLHVFNLLRDLDPHAIDPYAFGLVQLLCHWLAMSSACYNPFIYAWLHDSFREELRKLLVAWPRKIAPHGQNMTVSVVI

SEQ ID NO: 9 - protein, 306 aa, Homo sapiens
MLYSVVVVVGLVGNCLLVLVIARVRRLHNVTNFLIGNLALSDVLMCTACVPLTLAYAFEPRGWVFGGGLCHLVFFLQPVTVYVSVFTLTTIAVDRYVVLVHPLRRRISLRLSAYAVLAIWALSAVLALPAAVHTYHVELKPHDVRLCEEFWGSQERQRQLYAWGLLLVTYLLPLLVILLSYVRVSVKLRNRVVPGCVTQSQADWDRARRRRTFCLLVVVVVVFAVCWLPLHVFNLLRDLDPHAIDPYAFGLVQLLCHWLAMSSACYNPFIYAWLHDSFREELRKLLVAWPRKIAPHGQNMTVSVVI

SEQ ID NO: 10 - protein, 64 aa, Homo sapiens
MASSTTRGPRVSDLFSGLPPAVTTPANQSAEASAGNGSVAGADAPAVTPFQSLQLVHQLKGLIV

SEQ ID NO: 11 - DNA, 15 bp, Artificial
  GPR10 (TaqMan Probe 1)
cagtgaggtcatggc

SEQ ID NO: 12 - DNA, 15 bp, Artificial
  GPR10 (TaqMan Probe 2)
cagtgaggttatggc

SEQ ID NO: 13 - DNA, 20 bp, Artificial
  GPR10 (Forward Primer GR-F1)
ccaggagagaagttgggtga

SEQ ID NO: 14 - DNA, 19 bp, Artificial
  GPR10 (Reverse Primer GR-R1)
gacggcccagaaaacaaat

SEQ ID NO: 15 - DNA, 29 bp, Rattus sp.
atgaattcgtggccatgacctcactgccc

SEQ ID NO: 16 - DNA, 29 bp, Rattus sp.
atgaattcgtggccataacctcactgccc

SEQ ID NO: 17 - DNA, 30 bp, Rattus sp.
tagcggccgctcagatgaccacactgacgg

SEQ ID NO: 18 - DNA, 26 bp, Homo sapiens
cggaattcgtggccatggcctcatcg

SEQ ID NO: 19 - DNA, 29 bp, Homo sapiens
cgagcggccgctcagatgaccacgctgac

SEQ ID NO: 20 - DNA, 29 bp, Homo sapiens
gtgaattcatgctctacagcgtcgtggtg